(19) United States
(12) Patent Application Publication
(10) Pub. No.: US 2011/0256633 A1
(43) Pub. Date: Oct. 20, 2011

(54) Secretory Granules and Granulogenic Factors as a Target for Cancer Treatment

(75) Inventors: Seung Hyun Yoo, Incheon (KR); Yong Suk Hur, Incheon (KR)

(73) Assignee: INHA-INDUSTRY PARTNERSHIP INSTITUTE, Incheon (KR)

(21) Appl. No.: 12/740,160

(22) PCT Filed: Dec. 7, 2009
(86) PCT No.: PCT/KR09/07274

§ 371 Date: Jun. 6, 2011

(30) Foreign Application Priority Data
Jun. 18, 2009 (KR) 10-2009-0054342

(51) Int. Cl. G01N 33/574

(52) U.S. Cl. 436/501

(57) ABSTRACT

The present invention relates to a method for screening a cancer therapeutic agent, comprising the steps of: (a) contacting a test substance of interest with a cell containing a granulogenic factor-encoding nucleotide sequence; and (b) analyzing expression of the granulogenic factor or production of secretory granules, wherein the test substance is determined as the cancer therapeutic agent where it inhibits the expression of the granulogenic factor or the production of secretory granules. In the present invention, the expression of the granulogenic factor contributes to induction of secretory granule formation in non-secretory cells, and inhibition of the granulogenic factor expression leads to inhibition of secretory granule formation in secretory cells. In addition, the secretory granules produced by the present granulogenic factor change cell activities via the IP₃-dependent cellular Ca²⁺ regulatory mechanism, and the changes of cellular Ca²⁺ homeostasis will affect the development and proliferation of cancer cells. Therefore, a pharmaceutical composition containing as an active ingredient a substance which inhibits expression of a granulogenic factor gene, production of secretory granules, or activity of the granulogenic factor may be utilized in cancer prophylaxis or treatment, and also be used as a kit for identifying a cancer.

### BACKGROUND OF THE INVENTION

1. Field of the Invention

The present invention relates to biomarkers for cancer treatment and a screening method using the same, pharmaceutical compositions for cancer prophylaxis or treatment, and a kit for identifying cancers.

2. Description of the Related Art

Glial cells in the brain are thought to play essential roles in cellular communication not only among themselves but also with the neighboring neurons, thereby nurturing and nourishing the communication networks in the brain (Panatier et al. 2006; Montana et al. 2006; Angulo et al. 2004; Fellin et al. 2006; Perea and Araque 2005; Volterra and Meldolesi 2005; Haydon and Carmignoto 2006). The glial astrocytes are known to store and release a variety of signal molecules in a Ca²⁺-dependent regulated exocytotic pathway (Martineau et al. 2008; Bezzi et al. 2004; Coco et al. 2003; Krzan et al. 2003; Montana et al. 2004; Potokar et al. 2008; Parpura and Haydon 2000; Kreft et al. 2004; Santello and Volterra 2009). Hence the exocytotic secretory vesicles in astrocytes are viewed as essential in the cell-to-cell communication although the identity of molecules that are stored in different types of secretory vesicles in astrocytes is only partially known. There exist generally two types of secretory vesicles in astrocytes; one being the small transparent synaptic-like vesicles and the other the large dense-core vesicles (Bezzi et al. 2004; Crippa et al. 2006; Maienschein et al. 1999; Ramamoorthy and Whim 2008). Due to extensive studies in neurons the small translucent synaptic-like vesicles of astrocytes have attracted more attention than large dense-core vesicles in the past.

Nevertheless, the large dense-core vesicles of glial cell astrocytes have been shown to contain a number of molecules including ATP, glutamate, neuropeptides, and secretogranin II (Calegari et al. 1999; Coco et al. 2003; Chen et al. 2005; Ramamoorthy and Whim 2008; Striedinger et al. 2007). Along with chromogranins A (CGA) and B (CGB), which are two major members of the granin protein family and are prototypical marker proteins of secretory granules (Helle 2000; Montero-Hadjadje et al. 2008; Taupenot et al. 2003; Winkler and Fischer-Colbrie 1992; Huttner et al. 1991), secretogranin II (SgII) is a third member of the granin protein family and is also a typical secretory granule marker protein (Huttner et al. 1991). The presence of SgII in the large dense-core vesicles identifies the dense-core vesicles as typical secretory granules and demonstrates the presence of bona-fide secretory granules in astrocytes. Moreover, Bergmann glial cells have also been shown to contain chromogranin A (McAuliffe and Hess 1990). The molecules stored in the large dense-core vesicles including SgII and neuropeptide Y (NPY) were shown to be released in response to appropriate stimuli in a Ca²⁺-dependent manner (Chen et al. 2005; Coco et al. 2003; Ramamoorthy and Whim 2008; Striedinger et al. 2007), thus confirming participation of secretory granules in secretory function of astrocytes.

Not only do chromogranins A and B, and secretogranin II serve as marker proteins of secretory granules, they also function as high-capacity, low-affinity Ca²⁺ storage proteins, binding 30-93 molecules of Ca²⁺/mol with dissociation constants (Kd) of 1.5-4.0 mM (Yoo et al. 2001; Yoo and Albanesi 1991; Yoo et al, 2007). In secretory granules of bovine chromaffin cells, there exist 2-3 mM of the granin proteins, thereby enabling secretory granules to store ˜40 mM Ca²⁺ (Haigh et al. 1989; Hutton 1989). As a result, secretory granules are the subcellular organelle that contains the most calcium in all types of secretory cells. As is the case with other secretory cells, the increase in intracellular Ca²⁺ concentrations ([Ca²⁺]i) of astrocytes plays essential roles in the regulated exocytosis of active molecules from both the small synaptic-like vesicles and the large secretory granules, and the increase in [Ca²⁺]i is thought to be primarily contributed by the IP₃-dependent releases from intracellular stores (Araque et al. 2000; Hua et al. 2004; Jeremic et al 2001).

Interestingly, secretory granules also contain large amounts of the IP₃R/Ca²⁺ channels (Yoo et al. 2001), containing more than half the cellular IP₃R/Ca²⁺ channels present in chromaffin cells (Huh et al. 2005c). As a result secretory granules rapidly release Ca²⁺ in response to IP₃ (Gerasimenko et al. 1996;Nguyen et al. 1998;Yoo and Albanesi 1990), and function as the major IP₃-sensitive intracellular Ca²⁺ store in neuroendocrine cells (Huh et al. 2006;Huh et al. 2005b). The IP₃-dependent Ca²⁺ store role of secretory granules is now widely observed in many different types of secretory cells (Gerasimenko et al. 2006; Quesada et al. 2003; Quesada et al. 2001; Santodomingo et al. 2008; Srivastava et al. 1999; Xie et al. 2006).

Throughout this application, various patents and publications are referenced and citations are provided in parentheses. The disclosure of these patents and publications in their entities are hereby incorporated by references into this application in order to more fully describe this invention and the state of the art to which this invention pertains.

### DETAILED DESCRIPTION OF THIS INVETNION

The present inventors have done intensive studies to develop novel biomolecules for treating cancers. As results, we have discovered that the production of secretory granules could be inhibited by preventing (alleviating) expression of cellular granulogenic factors, for example the granin proteins (chromogranin and secretogranin), which could potentially lead to inhibition of the development and/or progression of secretory cell cancers including the brain cancers (e.g., glioblastoma multiforme).

Accordingly, it is an object of this invention to provide a method for screening a cancer therapeutic agent.

It is another object of this invention to provide a pharmaceutical composition for preventing or treating secretory cell cancers.

It is still another object to this invention to provide a kit for diagnosing secretory cell cancers.

It is still another object to this invention to provide a method for preventing or treating secretory cell cancers, comprising administrating to a subject a pharmaceutical composition comprising a pharmaceutically effective amount of a substance inhibiting expression of a granulogenic factor gene, production of secretory granules, or activity of a granulogenic factor.

It is further still another object to this invention to provide a method for identifying a cancer, comprising a binding agent specifically bound to a granulogenic factor.

Other objects and advantages of the present invention will become apparent from the following detailed description together with the appended claims and drawings.

In one aspect of this invention, there is provided a method for screening a cancer therapeutic agent, comprising the steps of: (a) contacting a test substance with a cell containing a nucleotide sequence encoding a granulogenic factor; and (b) analyzing expression of the granulogenic factor or production of secretory granules, wherein the test substance is determined as the cancer therapeutic agent where it inhibits the expression of the granulogenic factor or the production of secretory granules.

The present inventors have done intensive studies to develop novel biomolecules for treating cancers. As results, we have discovered that the production of secretory granules could be inhibited by preventing (alleviating) expression of cellular granulogenic factors, for example the granin proteins (chromogranin and secretogranin), which could potentially lead to inhibition of the development and/or progression of secretory cell cancers including the brain cancers (e.g., glioblastoma multiforme)

The granins (chromogranins or secretogranins) are a family of acidic proteins present in the secretory granules of a wide variety of endocrine and neuro-endocrine cells. It has been reported that the exact function(s) of these proteins seem to be the precursors of biologically active peptides and/or they may act as helper proteins in the packaging of peptide hormones and neuropeptides.

According to the present invention, the inhibition of cellular granin proteins may contribute to development of a cancer therapeutic agent by inhibiting the secretory granule production.

The present invention provides a method for screening a cancer therapeutic agent, including the steps of:

(a) contacting a test substance of interest with a cell containing a granulogenic factor-encoding nucleotide sequence; and

(b) analyzing expression of the granulogenic factor or production of secretory granules, wherein the test substance is determined as the cancer therapeutic agent where it inhibits the expression of the granulogenic factor or the production of secretory granules.

According to a preferable embodiment, the granulogenic factor of the present invention includes granin proteins, more preferably chromogranins or secretogranins, much more preferably chromogranin A (CGA), chromogranin B (CGB) or secretogranin II (SgII), still much more preferably chromogranin B or secretogranin II, and most preferably chromogranin B.

In the first step of the present screening method, the test substance of interest is incubated with cells containing a nucleotide sequence as a target of this invention. Cells containing the nucleotide sequence as a target of this invention are not particularly limited, and preferably include any of secretory cells, and more preferably nerve cells and endocrine cells. Preferably, the cells include primary cultured cells, established cell lines or tumor cells. Most preferably, cells containing the nucleotide sequence as a target of this invention are human glial cells. The term “test substance” used in the present screening method refers to a substance which is used in the screening to determine whether it affects an expression level of the present marker. The test substance screened by the present method may be chemical compounds, nucleotide, antisense-RNA, siRNA (small interference RNA) and natural extracts, but is not limited to these.

Next, the expression level of the present marker in the test substance-treated cells is measured. The measurement of expression amount may be performed as described below. As results, the test substance may be determined as the cancer therapeutic agent where it inhibits the expression of the nucleotide sequence encoding the marker of the present invention, or the production of secretory granules.

The measurement of changes in expression of a gene encoding a granulogenic factor may be carried out according to various methods known to those ordinarily skilled in the art, for example, using RT-PCR (Sambrook et al, Molecular Cloning. A Laboratory Manual, 3rd ed. Cold Spring Harbor Press (2001)), Northern blotting (Peter B. Kaufma et al., Molecular and Cellular Methods in Biology and Medicine, 102-108, CRC press), cDNA microarray hybridization (Sambrook et al, Molecular Cloning, A Laboratory Manual, 3rd ed. Cold Spring Harbor Press (2001)) or in situ hybridization (Sambrook et al, Molecular Cloning, A Laboratory Manual, 3rd ed. Cold Spring Harbor Press (2001)).

According to RT-PCR protocol, total RNA is extracted from the test substance-treated cells, and first cDNA is prepared using dT primer and reverse transcriptase. Then, PCR reaction is carried out using first cDNA as a template and a granulogenic factor-specific primer set. The granulogenic factor-specific primer set is a sequence involved in the nucleotide sequence illustrated in SEQ ID No:1, No:3, and No:5. The resulting products are separated by electrophoresis and the band patterns are analyzed to measure the expression changes of granulogenic factors.

The analysis for evaluating the expression amounts of granulogenic factor proteins may be conducted in accordance with immunoassay methods known to one skilled in the art. The immunoassay format includes, but is not limited to, immunostaining assay, radioimmunoassay, radioimmuno-precipitation, Western blot assay, immunoprecipitation, enzyme-linked immunosorbent assay (ELISA), capture-ELISA, inhibition or competition assay and sandwich assay.

The immunoassay or immunostaining procedures can be found in Enzyme Immunoassay, E. T. Maggio, ed., CRC Press, Boca Raton, Fla., 1980; Gaastra, W., Enzyme-linked immunosorbent assay (ELISA), in Methods in Molecular Biology, Vol. 1, Walker, J. M. ed., Humana Press, NJ, 1984; and Ed Harlow and David Lane, Using Antibodies, A Laboratory Manual, Cold Spring Harbor Laboratory Press, 1999, which are incorporated herein by reference.

For example, according to the radioimmunoassay method, the radioisotope (e.g., C¹⁴, I¹²⁵, P³² and S³⁵)) labeled antibody may be used to detect the marker of the present invention.

According to the ELISA method, the specific example of the present method may further comprise the steps of: (i) coating a surface of a solid substrate with a cell lysate of interest; (ii) incubating the cell lysate with an antibody to be analyzed as a primary antibody; (iii) incubating the resultant of step (ii) with a secondary antibody conjugated to an enzyme; and (iv) measuring the activity of the enzyme.

The solid substrate coated with the primary antibody is a hydrocarbon polymer (e.g., polystyrene and polypropylene), a glass, a metal or a gel, and most preferably, a microtiter plate.

The secondary antibody conjugated to an enzyme includes, but is not limited to, an enzyme catalyzing colorimetric, fluorometric, luminescence or infra-red reactions, for example, alkaline phosphatase, β-galactosidase, horseradish peroxidase, luciferase and cytochrome P₄₅₀. Where using alkaline phosphatase, bromochloroindolylphosphate (BCIP), nitro blue tetrazolium (NBT) and ECF (enhanced chemifluorescence) may be used as a substrate; in the case of using horseradish peroxidase, chloronaphtol, aminoethylcarbazol, diaminobenzidine, D-luciferin, lucigenin (bis-N-methylacridinium nitrate), resorufin benzyl ether, luminol, Amplex Red reagent (10-acetyl-3,7-dihydroxyphenoxazine, Pierce), HYR (p-phenylenediamine-HCl and pyrocatechol), TMB (3,3,5,5-tetramethylbenzidine), ABTS (2,2′-Azine-di[3-ethylbenzthiazoline sulfonate]), o-phenyldiamine (OPD) and naphtol/pyronin, glucose oxidase and tNBT (nitroblue tetrazolium) and m-PMS (phenzaine methosulfate) may be used as a substrate.

According to the capture-ELISA method, the specific example of the present method may comprise the steps of: (i) coating a surface of a solid substrate with an antibody of the present target as a capturing antibody; (ii) incubating the capturing antibody with a cell sample; (iii) incubating the resultant of step (ii) with a detecting antibody having a fluorescent label which reacts with the granulogenic factor protein specifically; and (iv) measuring the signal generated from the label.

The detecting antibody includes a substance generating a detectable signal. The signal-generating substance bound to antibody includes, but is not limited to, chemical (e.g., biotin), enzyme (alkaline phosphatase, β-galactosidase, horseradish peroxidase and Cytochrome P₄₅₀), radio-isotope (e.g., C¹⁴, I¹²⁵, P³² and S³⁵), fluorescent (e.g., fluoresin), luminescent, chemiluminescent and FRET (fluorescence resonance energy transfer) substances. Various methods for labels and labelings are described in Ed Harlow and David Lane, Using Antibodies:A Laboratory Manual, Cold Spring Harbor Laboratory Press, 1999.

The analysis for measuring the activity or the signal of final enzyme in the ELISA and capture-ELISA method may be carried out by various methods known to those skilled in the art. The signal detection permits to a quantitative or qualitative analysis of the present marker. For example, the signal of each biotin- and luciferase-labeled protein may be feasibly detected using streptavidin and luciferin.

According to a preferable embodiment, the inhibition of the granulogenic factor gene expression or inhibition of secretory granule production in the present invention leads to reduction of the expression of cellular granulogenic factor, or of the number of secretory granule per cell, or of secretory granule area per total cell area. In more detail, the number of secretory granules and the secretory granule area per total cell area in this invention increased in the brain cancer 30-fold and 42-fold, respectively, compared to those in the normal tissue (Table 3 and FIG. 5). Therefore, inhibition of granulogenic factor expression in the present invention will contribute to reduction in the number of secretory granules in the brain cancer cells, and change the IP₃-dependent cellular Ca²⁺ regulatory mechanism (Yoo, 2009), which could potentially lead to inhibition of cancer development and/or proliferation.

According to a preferable embodiment, the cancer of the present invention is selected from the group consisting of brain cancer, neuroendocrine cancer, stomach cancer, lung cancer, breast cancer, ovarian cancer, liver cancer, nasopharyngeal cancer, laryngeal cancer, pancreatic cancer, bladder cancer, adrenal cancer, colon cancer, colorectal cancer, cervical cancer, prostate cancer, bone cancer, skin cancer, thyroid cancer, parathyroid cancer and ureter cancer.

The neuroendocrine cancer of the present invention includes, but is not limited to, carcinoid, Merkel's cell tumor, gastrinoma, insulinoma, glucagonoma, VIPoma, PPoma, somatostatinoma, calcitoninoma, GHRHoma, neurotensinoma, ACTHoma, GRFoma, parathyroid hormone-related peptide tumor, neuroblastoma, pheochromocytoma (or pheochromocytoma), thyroid carcinoma, small cell lung cancer (SCLC), (lung) large cell neuroendocrine carcinoma, extra-pulmonary small cell carcinoma (ESCC or EPSCC), neuroendocrine carcinoma of the cervix, multiple endocrine neoplasia type 1 (MEN-1 or MEN1), multiple endocrine neoplasia type 2 (MEN-2 or MEN2), neurofibromatosis type 1, tuberous sclerosis, Von Hippel-Lindau disease, neuroendocrine tumor of pituitary gland or Carney's complex.

According to a preferable embodiment, the cancer of the present invention is secretory cell tumors, more preferably brain cancer, neuroendocrine cancer, ganglioglioma, pituitary adenoma, pancreatic cancer, adrenal cancer, breast cancer, uterine cancer or prostate cancer, and most preferably brain cancer.

In another aspect of this invention, there is provided a pharmaceutical composition for preventing or treating a cancer, comprising as an active ingredient a substance which inhibits an expression of a granulogenic factor gene, production of secretory granules, or activity of a granulogenic factor.

The present pharmaceutical composition may include chemical substances, nucleotides, antisense oligonucleotides, siRNAs or natural extracts as an active ingredient.

According to a preferable embodiment, the pharmaceutical composition of the present invention includes antisense oligonucleotides or siRNAs which are complementary to nucleotide sequences described in SEQ IDs NO:1, NO:3 and NO:5.

The term “antisense oligonucleotide” used herein is intended to refer to nucleic acids, preferably, DNA, RNA or its derivatives, that are complementary to the base sequences of a target mRNA, characterized in that they bind to the target mRNA and interfere its translation to protein. The antisense oligonucleotide of the present invention refers to DNA or RNA sequences which are complementary to the base sequences of chromogranin A (SEQ ID NO:1), chromogranin B (SEQ ID NO:3) and secretogranin II (SEQ ID NO:5) mRNA, characterized in that they bind to the chromogranin A, chromogranin B and secretogranin II mRNA and interfere their translation to protein, translocation into cytoplasm, or essential activities to other biological functions. The length of antisense nucleic acids is in a range of 6-100 nucleotides, preferably 8-60 nucleotides, and more preferably 10-40 nucleotides.

The antisense nucleic acids may be modified at above one or more positions of base, sugar or backbone (De Mesmaeker et al., Curr Opin Struct Biol., 5(3): 343-55 (1995)). The nucleic acid backbone may be modified by phosphothioate, phosphotriester, methyl phosphonate, single chain alkyl, cycloalkyl, single chain heteroatomic, heterocyclic bond between sugars, and so on. In addition, the antisense nucleic acids may include one or more substituted sugar moieties. The antisense nucleic acids may include a modified base. The modified base includes hypoxanthine, 6-methyladenine, 5-me pyrimidine (particularly, 5-methylcytosine), 5-hydrownethylcytosine (HMC), glycosyl HMC, gentobiosyl HMC, 2-aminoadenine, 2-thiouracil, 2-thiothymine, 5-bromouracil, 5-hydroxmethyluracil, 8-azaguanine, 7-deazaguanine, N6(6-aminohexyl)adenine, 2,6-diaminopurine, and so on. In addition, the antisense nucleic acids of this invention may be chemically linked to one or more moieties or conjugates which enhance the activities and cell adhesions of antisense nucleic acids. The moiety includes, but is not limited to, water-insoluble moieties such as cholesterol moiety, cholesteryl moiety, cholic acid, thioether, thiocholesterol, lipid chains, phospholipid, polyamine, polyethylene glycol chain, adamentan acetic acid, palmityl moiety, octadecylamine, hexylamino-carbonyl-oxycholesterol moiety, and so forth. Oligonucleotides containing the water-insoluble moieties and preparation methods thereof are well-known to those ordinarily skilled in the art (U.S. Pat. Nos. 5,138,045, 5,218,105 and 5,459,255). The modified nucleic acids may contribute to increase stability to a nuclease and enhance a binding affinity between antisense nucleic acids and mRNA targets.

The antisense oligonucleotides may be synthesized in a test tube according to a conventional method for administration to body or synthesized in vivo. RNA polymerase I is used in an example to synthesize oligonucleotides in a test tube. One example to prepare antisense RNA in vivo is to transcribe antisense RNA using a vector with an opposite origin of multiple cloning site (MCS). Preferably, the sequence of the antisense RNA includes a stop codon blocking translation into a peptide sequence.

The pharmaceutical composition includes siRNA which is complementary to a nucleotide sequence described in SEQ IDs NO:1, NO:3 and NO:5.

The term “siRNA” used herein refers to a nucleic acid that enables to mediate RNA interference or gene silencing (Reference: WO 00/44895, WO 01/36646, WO 99/32619, WO 01/29058, WO 99/07409 and WO 00/44914). The siRNA to inhibit expression of a target gene provides effective gene knock-down method or gene therapy method. It was been first in plants, insects, Drosophila melanogaster and parasites and recently has been used for mammalian cell researches (Degot S, et al. 2002; Degot S, et al. 2004; Ballut L, et al. 2005).

The siRNA of the present invention may consist of a sense RNA strand (having a sequence corresponding to chromogranin A and B, and secretogranin II mRNA sequence) and an antisense RNA strand (having a sequence complementary to chromogranin A and B, and secretogranin II mRNA sequence) placed at opposite position each other. According to another embodiment, the siRNA of the present invention may be a single-stranded structure comprising self-complementary sense and antisense strands.

The siRNA of this invention is not restricted to a RNA duplex of which two strands are completely paired and may comprise non-paired portion such as mismatched portion with non-complementary bases and bulge with no opposite bases. The overall length of the siRNA is 10-100 nucleotides, preferably, 15-80 nucleotides, and more preferably, 20-70 nucleotides.

The siRNA may comprise either blunt or cohesive end so long as it enables to silent the chromogranin A and B, and secretogranin II expression due to RNAi effect. The cohesive end may be prepared in 3′-end overhanging structure or 5′-end overhanging structure.

The siRNA molecule of the present invention may be constructed by inserting a short nucleotide sequence (e.g., about 5-15 nt) between self-complementary sense and antisense strands. The siRNA expressed forms a hair-pin structure by intramolecular hybridization, resulting in the formation of stem-and-loop structure. The stem-and-loop structure is processed in vitro or in vivo to generate active siRNA molecule mediating RNAi.

According to a preferable embodiment, the siRNA of the present invention includes a nucleotide sequence contained in the nucleotide sequence described in SEQ IDs NO:1, NO:3 and NO₅. According to the present invention, the expression of the granulogenic factor was reduced to a level of 10-20% compared to normal expression level depending on the treatment of granulogenic factor-siRNA to tumor cells (example: PC12 cells) (See, Table 5 and FIGS. 12-13).

The pharmaceutically acceptable carrier contained in the pharmaceutical composition of the present invention, which is commonly used in pharmaceutical formulations, but is not limited to, includes lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methylcellulose, methylhydroxy benzoate, propylhydroxy benzoate, talc, magnesium stearate, and mineral oils. The pharmaceutical composition according to the present invention may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

The pharmaceutical composition according to the present invention may be administered orally or parenterally, and preferably, administered parenterally, e.g., by intravenous, subcutaneous or local.

A suitable dosage amount of the pharmaceutical composition of the present invention may vary depending on pharmaceutical formulation methods, administration methods, the patient's age, body weight, sex, pathogenic state, diet, administration time, administration route, an excretion rate and sensitivity for a used pharmaceutical composition. Preferably, the pharmaceutical composition of the present invention may be administered with a daily dosage of 0.0001-100 mg/kg (body weight).

According to the conventional techniques known to those skilled in the art, the pharmaceutical composition according to the present invention may be formulated with pharmaceutically acceptable carrier and/or vehicle as described above, finally providing several forms including a unit dose form and a multi-dose form. Non-limiting examples of the formulations include, but not limited to, a solution, a suspension or an emulsion in oil or aqueous medium, an elixir, a powder, a granule, a tablet and a capsule, and may further comprise a dispersion agent or a stabilizer.

In still another aspect of this invention, there is provided a kit for identifying a cancer, comprising a binding agent specifically bound to a granulogenic factor.

In further still another aspect of this invention, there is provided a method for identifying a cancer, comprising a binding agent specifically bound to a granulogenic factor.

The molecular marker of this invention may be indicative of cancer development, progression and/or metastasis, and also used in diagnosis of brain cancer development, progression and/or metastasis.

The term “identifying a cancer” used herein includes the following matters: (a) to determine susceptibility of a subject to a particular disease or disorder; (b) to evaluate whether a subject has a particular disease or disorder; (c) to assess a prognosis of a subject suffering from a specific disease or disorder (e.g., identification of pre-metastatic or metastatic cancer conditions, determination of cancer stage, or investigation of cancer response to treatment); or (d) therametrics (e.g., monitoring conditions of a subject to provide an information to treatment efficacy).

The expression analysis of the granulogenic factor in the present invention may be carried out using hybridization in which the probe containing a sequence complementary to nucleotide sequences of the present targets is used.

The term “complementary” with reference to sequence used herein refers to a sequence having complementarity to the extent that the sequence hybridizes or anneals specifically with the nucleotide sequence of the granulogenic factor genes described above under certain hybridization or annealing conditions. In this regard, the term “complementary” used herein has different meaning from the term “perfectly complementary”. The primer or probe of this invention may include one or more mismatch base sequences where it is able to specifically hybridize with the above-described nucleotide sequences.

The term “primer” used herein means a single-stranded oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, i.e., in the presence of four different nucleoside triphosphates and a thermostable enzyme in an appropriate buffer and at a suitable temperature. The suitable length of primers will depend on many factors, including temperature, application and source of primer, generally, 15-30 nucleotides in length. In general, shorter primers need lower temperature to form stable hybridization duplexes to templates.

The sequences of primers are not required to have perfectly complementary sequence to templates. The sequences of primers may comprise some mismatches, so long as they can be hybridized with templates and serve as primers. Therefore, the primers of this invention are not required to have perfectly complementary sequence to the granulogenic factor genes as templates; it is sufficient that they have complementarity to the extent that they anneals specifically to the nucleotide sequence of the granulogenic factor gene for acting acting as a point of initiation of synthesis. The primer design may be conveniently performed with referring to the granulogenic factor gDNA or cDNA sequences, preferably, cDNA sequence. For instance, the primer design may be carried out using computer programs for primer design (e.g., PRIMER 3 program).

The term “probe” used herein refers to a linear oligomer of natural or modified monomers or linkages, including deoxyribonucleotides, ribonucleotides and the like, which is capable of specifically hybridizing with a target nucleotide sequence, whether occurring naturally or produced synthetically. The probe used in the present method may be prepared in the form of preferably single-stranded and oligodeoxyribonucleotide probe.

To prepare primers or probes, the nucleotide sequence of the present target may be found in the GenBank. For example, the nucleotide sequences of chromogranin A and B, and secretogranin II as the target of this invention are disclosed in GenBank Accession Nos. Gene Id 1113 (NM_{—}001819.2; SEQ ID NO:1), Gene Id 1114 (NM_{—}001819.2; SEQ ID NO:3) and Gene Id 7857 (NM_{—}003469.3; SEQ ID NO:5), respectively, and primers or probes may be designed by reference with the nucleotide sequences.

Using probes hybridizable with the targets of the present invention, brain cancer is diagnosed or detected by hybridization-based assay.

Labels linking to the probes may generate a signal to detect hybridization and bound to oligonucleotide. Suitable labels include fluorophores ((e.g., fluorescein), phycoerythrin, rhodamine, lissamine, Cy3 and Cy5 (Pharmacia)), chromophores, chemiluminescers, magnetic particles, radioisotopes (e.g., P³² and S³⁵), mass labels, electron dense particles, enzymes (e.g., alkaline phosphatase and horseradish peroxidase), cofactors, substrates for enzymes, heavy metals (e.g., gold), and haptens having specific binding partners, e.g., an antibody, streptavidin, biotin, digoxigenin and chelating group, but not limited to. Labeling is performed according to various methods known in the art, such as nick translation, random priming (Multiprime DNA labeling systems booklet, “Amersham” (1989)) and kination (Maxam & Gilbert, Methods in Enzymology, 65: 499 (1986)). The labels generate signal detectable by fluorescence, radioactivity, measurement of color development, mass measurement, X-ray diffraction or absorption, magnetic force, enzymatic activity, mass analysis, binding affinity, high frequency hybridization or nanocrystal.

The nucleic acid sample (preferably, cDNA) to be analyzed may be prepared using mRNA from various biosamples. The biosample is preferblay a brain cell. Instead of probes, cDNA may be labeled for hyribridization-based analysis.

Probes are hybridized with cDNA molecules under stringent conditions for detecting a brain cancer. Suitable hybridization conditions may be routinely determined by optimization procedures known to those skilled in the art for setting up of protocols to be performed in the laboratory. Conditions such as temperature, concentration of components, hybridization and washing times, buffer components, and their pH and ionic strength may be varied depending on various factors, including the length and GC content of probes and target nucleotide sequence. The detailed conditions for hybridization can be found in Joseph Sambrook, et al., Molecular Cloning, A Laboratory Manual, Cold Spring Harbor Laboratory Press, Cold Spring Harbor, N.Y. (2001); and M.L.M. Anderson, Nucleic Acid Hybridization, Springer-Verlag New York Inc. N.Y. (1999). For example, the high stringent condition includes hybridization in 0.5 M NaHPO₄, 7% SDS (sodium dodecyl sulfate) and 1 mM EDTA at 65° C. and washing in 0.1×SSC (standard saline citrate)/0.1% SDS at 68° C. Also, the high stringent condition includes washing in 6×SSC/0.05% sodium pyrophosphate at 48° C. The low stringent condition includes, e.g., washing in 0.2×SSC/0.1% SDS at 42° C.

Following hybridization reactions, a hybridization signal indicative of the occurrence of hybridization is then measured. The hybridization signal may be analyzed by a variety of methods depending on labels. For example, where probes are labeled with enzymes, the occurrence of hybridization may be detected by reacting substrates for enzymes with hybridization resultants. The enzyme/substrate pair useful in this invention includes, but is not limited to, a pair of peroxidase (e.g., horseradish peroxidase) and chloronaphtol, aminoethylcarbazol, diaminobenzidine, D-luciferin, lucigenin (bis-N-methylacridinium nitrate), resorufin benzyl ether, luminol, Amplex Red reagent (10-acetyl-3,7-dihydro>cyphenoxazine), HYR (p-phenylenediamine-HCl and pyrocatechol), TMB (3,3,5,5-tetramethylbenzidine), ABTS (2,2-Azine-di[3-ethylbenzthiazoline sulfonate]), o-phenylenediamine (OPD) or naphtol/pyronine; a pair of alkaline phosphatase and bromochloroindolylphosphate (BCIP), nitro blue tetrazolium (NBT), naphthol-AS-B1-phosphate or ECF substrate; and a pair of glucosidase and t-NBT (nitroblue tetrazolium) or m-PMS (phenzaine methosulfate). Where probes are labeled with gold particles, the occurrence of hybridization may be detected by silver staining method using silver nitrate. In these connections, where the present method for diagnosing a brain cancer is carried out by hybridization, it comprises the steps of (i) contacting a nucleic acid sample to a probe having a nucleotide sequence complementary to the nucleotide sequence of the target of this invention as set forth in SEQ IDs NO:1, NO:3 and NO:5; and (ii) detecting the occurrence of hybridization. The signal intensity from hybridization is indicative of cancer/metastasis. When the hybridization signal to the target of this invention from a sample to be diagnosed is measured to be stronger than normal samples (e.g., brain tissue samples), the sample can be determined to have cancer/metastasis.

Where the diagnosing kit of this invention is performed using the protein, it also could be carried out according to conventional immunoassay procedures, i.e., antigen-antibody reaction. The diagnosing kit may be constructed by incorporating an antibody or aptamer binding to the target protein of this invention specifically.

The antibody against the target protein used in this invention may be polyclonal or monoclonal, preferably monoclonal. The antibody could be prepared according to conventional techniques such as a fusion method (Kohler and Milstein, European Journal of Immunology, 6: 511-519 (1976)), a recombinant DNA method (U.S. Pat. No. 4,816,56) or a phage antibody library (Clackson et al, Nature, 352: 624-628 (1991) and Marks et al, J. Mol. Biol., 222:58, 1-597 (1991)). The general procedures for antibody production are described in Harlow, E. and Lane, D., Using Antibodies: A Laboratory Manual, Cold Spring Harbor Press, New York, 1988; Zola, H., Monoclonal Antibodies: A Manual of Techniques, CRC Press, Inc., Boca Raton, Fla., 1984; and Coligan, CURRENT PROTOCOLS IN IMMUNOLOGY, Wiley/Greene, NY, 1991, which are incorporated herein by references. For example, the preparation of hybridoma cell lines for monoclonal antibody production is done by fusion of an immortal cell line and the antibody producing lymphocytes. This can be done by techniques well known in the art. Polyclonal antibodies may be prepared by injection of the target protein antigen to suitable animal, collecting antiserum containing antibodies from the animal, and isolating specific antibodies by any of the known affinity techniques.

Where the diagnosing method of this invention is performed using antibodies or aptamers to the target protein, it also could be carried out according to the described-above conventional immunoassay procedures for detecting brain cancer.

To prepare antibodies or aptamers, the amino acid sequence of the present target may be found in the GenBank. For example, the amino acid sequences of chromogranin A and B, and secretogranin II as the markers of this invention are disclosed in GenBank Accession Nos. Gene Id 1113 (NP_{—}001266.1; SEQ ID NO:2), Gene Id 1114 (NP_{—}001810.2; SEQ ID NO:4) and Gene Id 7857 (NP_{—}003460.2; SEQ ID NO:6), respectively, and thus antibodies or aptamers may be designed by reference with the amino acid sequences.

According to another modification of this invention, aptamer having a specific binding affinity to the target of the present invention may be used instead of antibody. The term “aptamer” used herein means an oligonucleic acid or peptide molecule, and general descriptions of aptamer are disclosed in Bock L C et al., Nature 355(6360):564-6 (1992); Hoppe-Seyler F, Butz K “Peptide aptamers: powerful new tools for molecular medicine”. J Mol Med. 78 (8): 426-30 (2000); and Cohen B A, Colas P, Brent R. “An artificial cell-cycle inhibitor isolated from a combinatorial library”. Proc Nati Acact Sci USA. 95 (24): 14272-7 (1998).

The final signal intensity measured by the above-mentioned immunoassay procedures is indicative of cancer/metastasis. When the signal to the target of this invention from a sample to be diagnosed is stronger than normal samples (e.g., glioblastoma multiforme), the sample can be diagnosed as cancer/metastasis.

The kit of the present invention may optionally include other reagents along with primers, probes or antibodies described above. For instance, where the present kit may be used for nucleic acid amplification, it may optionally include the reagents required for performing PCR reactions such as buffers, DNA polymerase (thermostable DNA polymerase obtained from Thermus aquaticus (Taq), Thermus thermophllus (Tth), Thermus Thermis flavus, Thermococcus literalis, and Pyrococcus furiosus (Pfu)), DNA polymerase cofactors, and deoxyribonucleotide-5-triphosphates. The kits, typically, are adapted to contain in separate packaging or compartments the constituents afore-described.

The target of the present invention is biomolecules highly expressed in cancer/metastasis. The high expression of markers may be measured at mRNA or protein level. The term “high expression” used herein with reference to cancer/metastasis means that the nucleotide sequence of interest in a sample to be analyzed is much more highly expressed than that in the normal sample, for instance, a case analyzed as high expression according to analysis methods known to those skilled in the art, e.g., RT-PCR method or ELISA method (See, Sambrook, 3. et al., Molecular Cloning. A Laboratory Manual, 3rd ed. Cold Spring Harbor Press (2001)). Using analysis methods as described above, where the markers of the present invention are much more highly expressed at a range of 2-80 fold (at average, 7.6-10.5 fold) in cancer cells than in normal cells, this case is determined as “high expression” and diagnosed as cancer/metastasis in the present invention (See, FIGS. 6-7).

In still another aspect of this invention, there is provided a method for preventing or treating a cancer, comprising administrating to a subject a pharmaceutical composition comprising a pharmaceutically effective amount of a substance inhibiting an expression of a granulogenic factor gene, production of secretory granules, or activity of a granulogenic factor.

In further still another aspect of this invention, there is provided a method for identifying a cancer, comprising a binding agent specifically bound to a granulogenic factor.

Since the present method comprises the granulogenic factor of this invention as active ingredients described above, the common descriptions between them are omitted in order to avoid undue redundancy leading to the complexity of this specification.

As described above, the high expression of the granulogenic factor of this invention leads to significant increases in the number and area of secretory granules in cancer cells (example: glioblastoma multiforme) (Reference: FIGS. 5-7), suggesting that the granulogenic factor plays an important role in the production of secretory granules in cancer cells, and administration of the present composition to a cancer (particularly, brain cancer) subject may contribute to inhibition of cancer development and proliferation through the IP₃-dependent cellular Ca²⁺ regulatory mechanism (Yoo, 2009). Therefore, the pharmaceutical composition of this invention may be utilized in prevention or treatment of cancer, and also used as a kit for diagnosing a cancer.

The features and advantages of this invention are summarized as follows:

(a) The present invention provides a method for screening a cancer therapeutic agent using a granulogenic factor.

(b) In the present invention, the expression of the granulogenic factor contributes to induction of secretory granule formation in non-secretory cells, and inhibition of the granulogenic factor expression leads to inhibition of secretory granule formation in secretory cells.

(c) The secretory granules produced by the present granulogenic factor change cell activities via the IP₃-dependent cellular Ca²⁺ regulatory mechanism, and the changes of cellular Ca²⁺ homeostasis will affect the development and proliferation of cancer cells.

(d) Therefore, a pharmaceutical composition containing as an active ingredient a substance which inhibits expression of a granulogenic factor gene, production of secretory granules, or activity of a granulogenic factor may be utilized in cancer prophylaxis or treatment, and also be used as a kit for identifying a cancer.

### BRIEF DESCRIPTION OF THE DRAWINGS

FIG. 1 represents electron micrographs of the secretory granule-like vesicles (large dense-core vesicles) in astrocytes of normal brain tissues. Normal human brain tissues are examined by electron microscope and secretory granule-like vesicles (large dense-core vesicles) in the cell body (A) and cell process (B) of astrocytes are shown. Secretory granule-like vesicles (SG) are indicated by arrows. Nu, nucleus; M, mitochondria; ax, axon; fm, filament. Bar=200 nm.

FIG. 2 shows immunogold electron micrographs of the localization of CGB and SgII in secretory granule-like vesicles (large dense-core vesicles) in astrocytes of normal brain tissues. Astrocytes from normal human brain tissues were immunolabeled for CGB (A) and SgII (B) (15 nm gold) with the affinity purified CGB and SgII antibodies, respectively. Secretory granule-like vesicles (SG) are indicated by arrows. The CGB- or SgII-labeling gold particles are primarily localized in the secretory granule-like vesicles with some in the endoplasmic reticulum (er), but not in the mitochondria (M). In the control experiments without the primary antibody no gold particles were seen in the secretory granule-like vesicles (not shown). Bar=200 nm.

FIG. 3 represents electron micrographs of the secretory granules in astrocytes of glioblastoma multiforme brain tissues. Glioblastoma multiforme brain tissues are examined by electron microscope and secretory granules in the cell body (A) and cell process (B) of astrocytes are shown. Secretory granules (SG) and mitochondria (M) are indicated by different arrows. Nu, nucleus; er, endoplasmic reticulum. Bar=200 nm.

FIG. 4 is immunogold electron micrographs showing the localization of CGB and SgII in secretory granules in astrocytes of glioblastoma multiforme brain tissues. Astrocytes from GBM tissues were immunolabeled for CGB (A) and SgII (B) (15 nm gold) with the affinity purified CGB and SgII antibodies, respectively. Secretory granules (SG) and mitochondria (M) are indicated by closed arrows. The CGB-(A) or SgII-labeling (B) gold particles are primarily localized in secretory granules (indicated with open arrows) with some in the endoplasmic reticulum (er), but not in the mitochondria (M). In the control experiments without the primary antibody no gold particles were seen in secretory granules (not shown). Bar=200 nm.

FIG. 5 represents distribution of secretory granules in astrocytes of normal and glioblastoma multiforme human brain tissues. The number of and the area occupied by secretory granules in astrocytes of normal and GBM brain tissues are expressed (mean±s.e.) in a bar graph along with the paired t-test results. The number of secretory granules per cell image (left side) and the area occupied by secretory granules over the total cell image area (right side, %) are shown.

FIG. 6 is immunoblot analysis of chromogranin B expression in the protein extracts from normal and GBM brain tissues. The protein extracts from each of the six different normal (N-1-N6) and GBM (G1-G6) brain tissues were resolved on a 10% SDS-polyacrylamide gel and analyzed by immunoblot using the affinity purified CGB antibody (A). The immunoblot result is shown in the top panel, and the bar graph showing the result of densitometric analysis of the immunoblot is shown in the bottom panel. The CGB expression levels, as determined from the densitometric results, in both the normal and GBM tissues are expressed (mean±s.e.) in a bar graph along with the paired t-test result (B).

FIG. 7 represents immunoblot analysis of secretogranin II expression in the protein extracts from normal and GBM brain tissues. The protein extracts from each of the six different normal (N-1-N6) and GBM (G1-G6) brain tissues were resolved on a 10% SDS-polyacrylamide gel and analyzed by immunoblot using the affinity purified SgII antibody (A). The immunoblot result is shown in the top panel, and the bar graph showing the result of densitometric analysis of the immunoblot is shown in the bottom panel. The SgII expression levels, as determined from the densitometric results, in both the normal and GBM tissues are expressed (mean±s.e.) in a bar graph along with the paired t-test result (B).

FIG. 8 is electron micrographs showing the newly formed dense-core granules in non-neuroendocrine NIH3T3 cells. Non-neuroendocrine NIH3T3 cells were transfected with pCI-CGA or CGB, and appearance of the newly formed dense-core granules was examined by electron microscopy. Normal NIH3T3 cells (A), CGA- (B) and CGB-transfected (C), and empty vector-transfected cells (D). Several of the newly formed dense-core granules are indicated by arrows (large arrow head, large granule; small arrow, small granule). Nu, nucleus; M, mitochondria; G, Golgi; er, endoplasmic reticulum. Bar=200 nm.

FIG. 9 represents electron micrographs of the newly formed dense-core granules in non-neuroendocrine COS-7 cells. Non-neuroendocrine COS-7 cells were transfected with pCI-CGA or -CGB, and the appearance of newly formed dense-core granules was examined by electron microscopy. CGA- (A) and CGB-transfected (B), and empty vector-transfected (C)COS-7 cells. Several of the newly formed dense-core granules are indicated by arrows (large arrow head, large granule; small arrow, small granule). M, mitochondria; G, Golgi; er, endoplasmic reticulum. Bar=200 nm.

FIG. 10 represents expression of bovine CGA and CGB in transiently transfected NIH3T3 cells. The total protein extracts from the NIH3T3 cells transfected with pCI-CGA (A) or -CGB (B) were resolved on 10% SDS-gels, and probed with the anti-CGA or CGB antibody. The blots were also reprobed with the α-tubulin antibody after deprobing the first blots to check the amount of proteins loaded. The protein extracts from both the untransfected (normal) and the pCI-neo vector-transfected (pCI-empty) cells were used as controls.

FIG. 11 is immunogold electron micrographs showing the localization of CGA and CGB in the newly formed secretory granules of NIH3T3 and COS-7 cells. The NIH3T3 cells transfected with CGA or CGB were immunolabeled for CGA (A) and CGB (B) (10 nm gold) with the affinity purified CGA and CGB antibodies, respectively. The COS-7 cells transfected with CGB were also immunolabeled for CGB (C) (10 nm gold). Several of the newly formed secretory granules are indicated by arrows (large arrow head, large granule; small arrow, small granule). The CGA- or CGB-labeling gold particles are primarily localized in the secretory granules with some in the endoplasmic reticulum (er), but not in the mitochondria (M). In the control experiments without the primary antibody no gold particles were seen in the secretory granules (not shown). Bar=200 nm.

FIG. 12 is electron micrographs showing secretory granules in neuroendocrine PC12 cells. Normal neuroendocrine PC12 cells contain a number of intrinsic secretory granules (A). However, the cells transfected with CGA- (B) or CGB-siRNA (C) contained markedly reduced number of secretory granules, whereas the cells transfected with the same reagents, but without the siRNA (D), contained the same number of secretory granules. Several secretory granules are indicated by arrows. Nu, nucleus; M, mitochondria; G, Golgi; er, endoplasmic reticulum. Bar=200 nm.

FIG. 13 represents inhibition of the expression of chromogranins A and B by CGA- and CGB-siRNAs in PC12 cells. The indicated amounts of CGA- (A) or CGB-siRNA (B) were transfected into 5×10⁵ PC12 cells, and the expression levels of CGA and CGB were analyzed by immunoblot analysis 48 h after transfection. The expressed proteins were analyzed using CGA- (left panel) and CGB-specific (right panel) antibodies for the CGA-siRNA treated cells (A), and using CGB- (left) and CGA-specific (right) antibodies for the CGB-siRNA treated cells (B). The same blots were reprobed with the α-tubulin antibody after deprobing.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Experimental Procedures

### Antibodies

The polyclonal anti-rabbit CGB antibody was raised against purified intact bovine CGB (Yoo 1995), and affinity purified against bovine recombinant CGB (Yoo et al. 2007). The specificity of the antibody was confirmed (Park et al. 2002; Huh et al. 2003; Yoo et al. 2002; Yoo et al. 2001). Monoclonal SgII antibody production was carried out with the secretory vesicle lysate proteins from bovine adrenal chromaffin cells as described previously (Park et al. 2002).

### Human Tissue Samples

All the brain tissue samples examined in this study were obtained from patients undergoing surgical treatments following written consent in accordance with appropriate clinical protocols and were histologically diagnosed as glioblastoma multiforme (grade IV) according to WHO classification.

Extraction of Proteins from Brain Tissues and Immunoblot Analysis

To obtain the total protein extracts from the brain tissues, the samples that had been kept frozen at −80° C. were thawed and mixed with a lysis buffer (50 mM Tris-HCl, pH 8.0, 150 mM NaCl, 0.02% sodium azide, 1% NP-40, 0.5% deoxycholate, 0.1% SDS, 1 mM phenylmethylsulfonyl fluoride, and 20 μg/ml aprotinin/leupeptin mix) twice the volume of the sample. The tissues were then thoroughly homogenized, followed by sonication for 10 min on ice. After incubation for 20 min on ice, the tissue debris was removed by centrifugation at 21,000×g for 30 min at 4° C., and the supernatant was used as the protein extracts. The proteins (40 μg each) were then resolved by SDS-PAGE and subjected to immunoblot analysis using the appropriate antibodies and an image detection system (UVP Bioimaging system).

### Immunogold Electron Microscopy

For the electron microscopic study of human brain tissues, both the normal and GBM brain tissues were minced into small pieces (˜1 mm³) and fixed for 2 h at 4° C. in PBS containing 0.1% glutaraldehyde, 4% paraformaldehyde immediately after surgical removal. After three washes in PBS, the tissues were postfixed with 1% osmium tetroxide on ice for 2 h, and washed three times in PBS. The tissues were then embedded in Epon 812 after dehydration in an ethanol series. After collection of the ultrathin (70 nm) sections on Formvar/carbon-coated nickel grids, the grids were stained with 2.5% uranyl acetate (7 min) and lead citrate (2 min).

For immunogold labeling experiments, the ultrathin sections that had been collected on Formvar/carbon-coated nickel grids were floated on drops of freshly prepared 3% sodium metaperiodate for 40 min. After etching and washing, the grids were placed on 50 μl droplets of buffer A (phosphate saline solution, pH 8.2, containing 4% normal goat serum, 1% BSA, 0.1% Tween 20, 0.1% sodium azide) for 1 h. After an extensive washing in buffer A, the grids were then incubated for 3 h at room temperature in a humidified chamber on 50 μl droplets of polyclonal anti-rabbit CGB or monoclonal anti-mouse SgII antibody appropriately diluted in solution B (solution A but with 1% normal goat serum), followed by rinses in solution B. The grids were reacted with the 15-nm gold-conjugated goat anti-rabbit or anti-mouse IgG, diluted in solution A. Controls for the specificity of CGB- or SgII-specific immunogold labeling included 1) omitting the primary antibody, 2) replacing the primary antibody with the preimmune serum, and 3) adding the primary antibody in the excess presence of purified CGB or SgII. After washes in PBS and deionized water, the grids were stained with uranyl acetate (7 min) and lead citrate (2 min). Following washing in deionized water and drying the samples were examined with a JEOL JEM-1011 electron microscope.

### Construction of Expression Vectors

The expression vectors for CGA and CGB were prepared by polymerase chain reaction (PCR) using bovine cDNA as a template, and the PCR products containing full coding sequences were subcloned into EcoRI/XbaI site of pCI-neo mammalian expression vector (Promega), in which transcription of the cloned gene is under the direction of the constitutively active cytomegalovirus promoter. Circular plasmid cDNAs for transfection were prepared using Qiagen maxi-preparation kit.

### NIH3T3, COS-7 Cell Culture and Transient Transfection

All culture reagents and powdered media were purchased from GibcoBRL. COS-7 and NIH3T3 cells were maintained in Dulbecco's modified Eagle's medium (DMEM) supplemented with 10% fetal bovine serum. Transient transfection was performed with 70-80% confluent cultures. The cells were transfected with circular plasmid DNAs using LipofecTAMINE-plus transfection reagent (GibcoBRL). Briefly, cells were plated at a density of 5×10⁵ cells per well (100-mm in diameter), and were cultured for additional 24 h. Four pg of plasmid DNA in 20 μl of LipofecTAMINE plus reagent were mixed with 750 μl of OPTI-MEM I medium and incubated for 15 min at room temperature. In addition, 30 μl of LipofecTAMINE reagent was mixed with 750 μl of OPTI-MEM I and incubated for 15 min. The mixture was then added into a culture plate containing 5 ml OPTI-MEM I medium. The transfection was performed for 3 h at 37° C. After transfection, the medium was replaced with fresh pre-warmed culture medium, and was further incubated for 72 h. In our culture condition, about 40-50% of COS-7 and 70-80% of NIH3T3 cells were transfected. The pCI-neo vector was used as an empty vector.

### PC12 Cell Culture and Transient Transfection of CGA- and CGB-siRNAs

PC12 cells were maintained in RPMI 1640 (Gibco BRL) medium supplemented with 10% fetal bovine serum. Transient siRNA transfection was performed with 70-80% confluent cultures. The CGA-siRNA duplex sense and antisense sequences are 5′-CAACAACAACACAGCAGCUdTdT-3′ and 3′-dTdTGUUGUUGUUGUGUCGUC GA -5′, respectively, and the CGB-siRNA duplex sense and antisense sequences are 5′-AUGCCCUAUCCAAGUCCAGdTdT-3′ and 3′-dTdTUACGGGAUAGGUUCAGGUC-5′, respectively. The 2-nucleotide 3′-overhang of 2′-deoxythymidine is indicated as dTdT. The cells were transfected with the siRNAs using Silencer™ siRNA transfection kit (Ambion). Briefly, approximately 1−2×10⁶ PC12 cells were plated on collagen type IV (BD Biosciences) coated culture dish (100 mm in diameter) in RPMI 1640 medium supplemented with 10% FBS and were cultured for 48 h before transfection. For dose-response experiments of siRNA transfection, 0.25-2 pg of appropriate siRNA and 10 μl siPORT Amine were used per 5×10⁵ cells. But for the EM study, 1 μg of appropriate siRNA and 10 μl siPORT Amine were used per 5×10⁵ cells. Addition of more siRNA did not reduce the number of secretory granules further. The transfection was performed for 6 h at 37° C. After transfection, the medium was replaced with fresh pre-warmed RPMI 1640 medium, and was further incubated for 48 h. The transfection was monitored using Silencer CyTM3 siRNA Labeling Kit, and the electron microscope experiments using the transfected PC12 cells were performed 48 h after transfection.

### Results

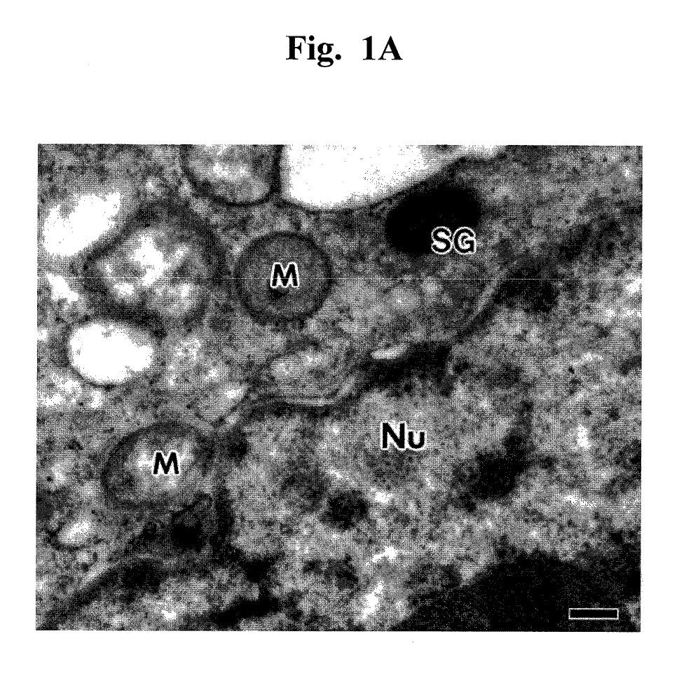

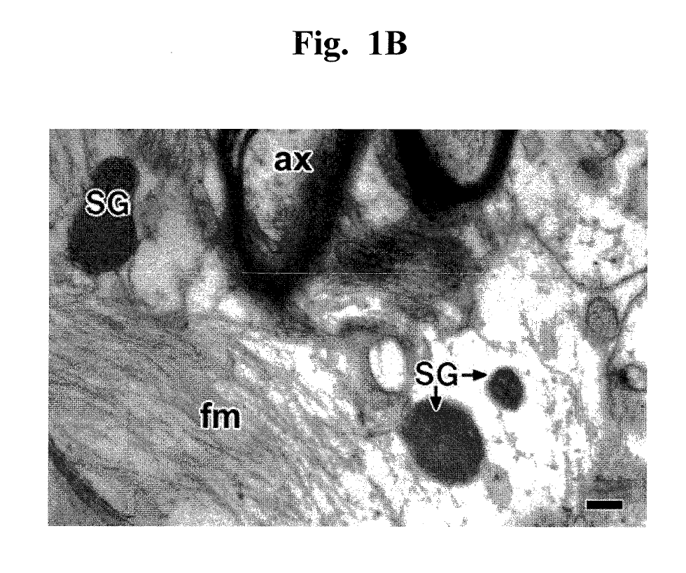

In our attempt to find the basis for differences between the normal and GBM brain tissues, we have examined and compared the brain tissue samples from the cancerous and noncancerous regions (obtained by lobectomy) of the brains by electron microscopy. As shown in FIG. 1, in astrocytes of normal brain tissues we could normally observe 0-3 secretory granule-like vesicles (large dense-core vesicles) in one picture image that encloses the cell body of an astrocyte (FIG. 1A), but occasionally we could observe 2-3 secretory granule-like vesicles in an image of a cell process (FIG. 1B), giving the impression that the secretory granule-like vesicles are more likely to be found in the cell processes than in the cell body of normal astrocytes.

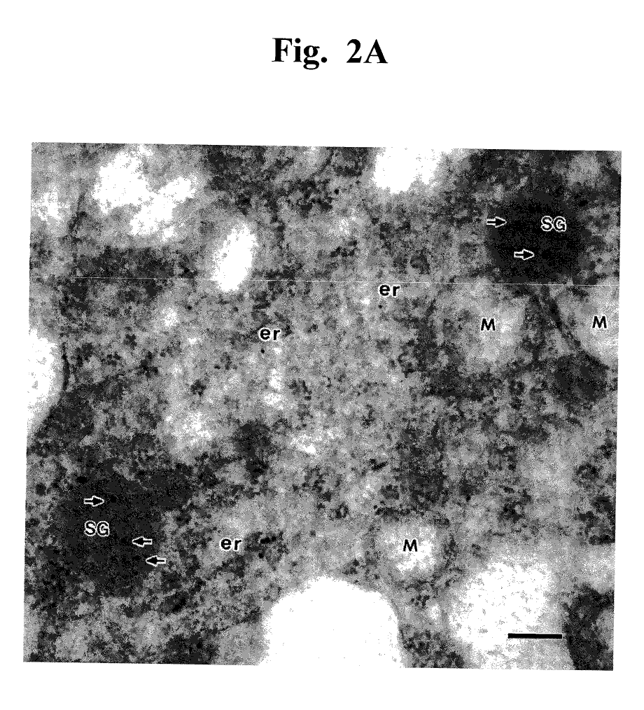

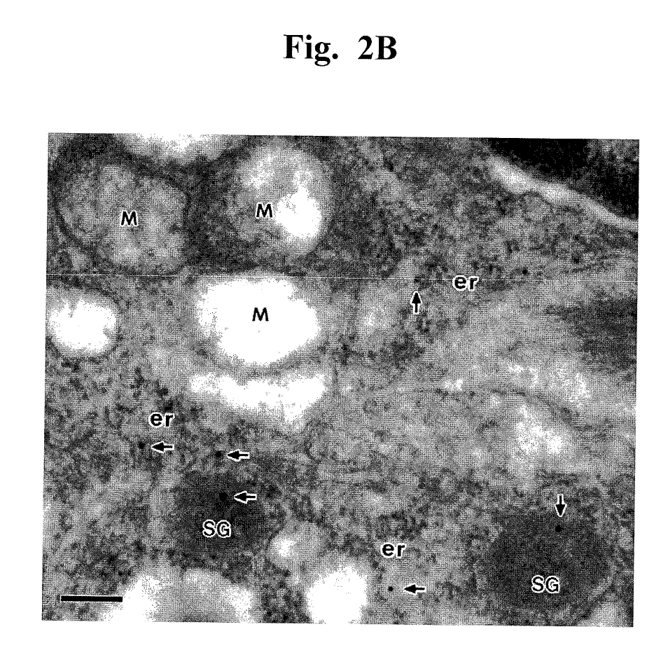

To determine the identity of the secretory granule-like vesicles in astrocytes, we have investigated the potential localization of secretory granule marker proteins chromogranin B and secretogranin II in the secretory granule-like vesicles by immunogold electron microscopy using antibodies specific for chromogranin B (FIG. 2A) and secretogranin II (FIG. 2B). Chromogranins A and B, and secretogranin II are granulogenic factors that induce formation of secretory granules in the cells they are expressed (Beuret et al. 2004; Huh et al. 2003; Kim et al. 2001). Hence, the granin proteins are found in secretory granules of virtually all types of secretory cells, thus entitling them as secretory granule marker proteins (Huttner et al. 1991). Although chromogranins A and B are two major members of the granin protein family (Helle 2000; Montero-Hadjadje et al. 2008; Taupenot et al. 2003; Winkler and Fischer-Colbrie 1992; Huttner et al. 1991), chromogranin B is more abundant in secretory granules of humans.

As shown in FIG. 2A, chromogranin B was present in the secretory granule-like vesicles in addition to its localization in the endoplasmic reticulum (ER). Being a secretory protein CGB localizes to the ER before traveling to the Golgi and on to secretory granules, but it is known to be absent in mitochondria (Huh et al. 2005a). Consistent with the previous results that showed absence of the granin proteins in mitochondria (Huh et al. 2005a; Huh et al. 2003; Huttner et al. 1991; Winkler and Fischer-Colbrie 1992), chromogranin B was absent in mitochondria. The expression of CGB in the secretory granule-like vesicles confirms that these large dense core vesicles are bona fide secretory granules. The chromogranin-containing secretory granules in secretory cells are usually large, with sizes varying from 200 nm to 500 nm in diameter, but with an average diameter of ˜300 nm (Huh et al. 2005a; Coupland 1968). Likewise, the average diameter of secretory granules of normal astrocytes appeared to be ˜300 nm, which is consistent with the results shown in other study (Chen et al. 2005). In addition, another secretory granule marker protein secretogranin II was also expressed in the secretory granule-like vesicles (FIG. 2B), confirming the identity of the secretory granule-like vesicles as secretory granules. Secretogranin II also localized to the ER as expected but was absent in mitochondria consistent with the previous results (Park et al, 2002). The relative abundance of CGB- and SgII-labeling gold particles per unit area of secretory granules compared to that of the ER (cf. Tables 1 and 2) suggests expression of relatively large amounts of CGB and SgII in secretory granules of astrocytes.

TABLE 1
Distribution of chromogranin B-labeling gold particles in astrocytes
of normal and glioblastoma multiforme human brain tissues.

| | | Number | Area | |
|---|---|---|---|---|
| | | of gold | viewed | Number of gold |
| Cell | Organelle | particles | (μm²) | particles per μm² |
| Normal^{a} | Secretory granules | 96 | 7.752 | 12.38 |
| | Mitochondria | 9 | 24.082 | 0.37 |
| GBM^{b} | Secretory granules | 345 | 28.779 | 11.98 |
| | Mitochondria | 9 | 24.661 | 0.36 |

^{a}39 images from three different tissues were used.
^{b}44 images from three different tisses were used.

TABLE 2
Distribution of secretogranin II-labeling gold particles in
astrocytes of normal and glioblastoma human brain tissues.

| | | Number | Area | |
|---|---|---|---|---|
| | | of gold | viewed | Number of gold |
| Cell | Organelle | particles | (μm²) | particles per μm² |
| Normal^{a} | Secretory granules | 74 | 7.174 | 10.31 |
| | Mitochondria | 8 | 20.450 | 0.39 |
| GBM^{b} | Secretory granules | 283 | 32.343 | 8.70 |
| | Mitochondria | 9 | 29.343 | 0.31 |

³42 images from three different tissues were used.
^{b}49 images from three different tisses were used.

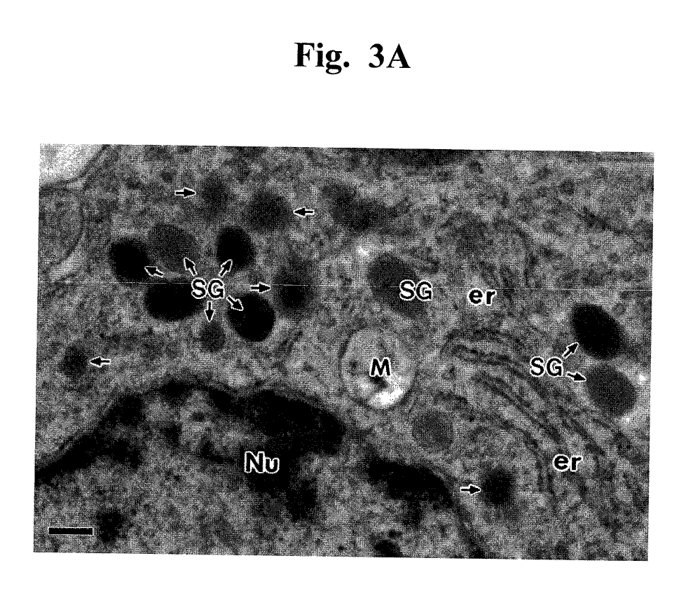

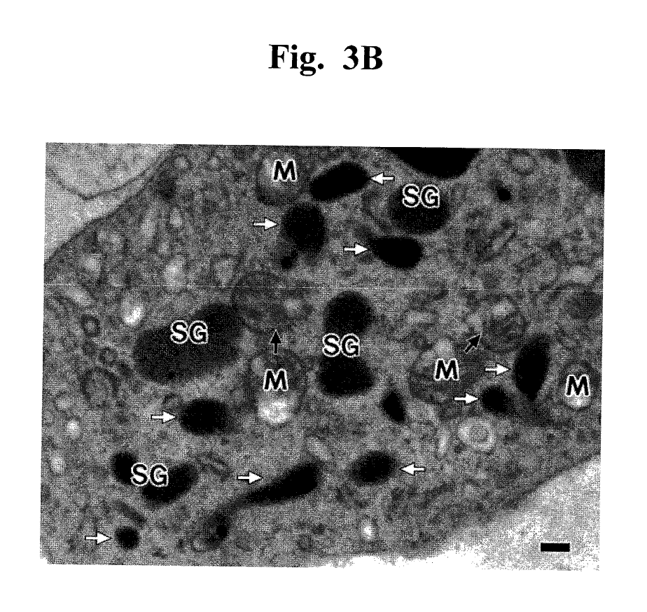

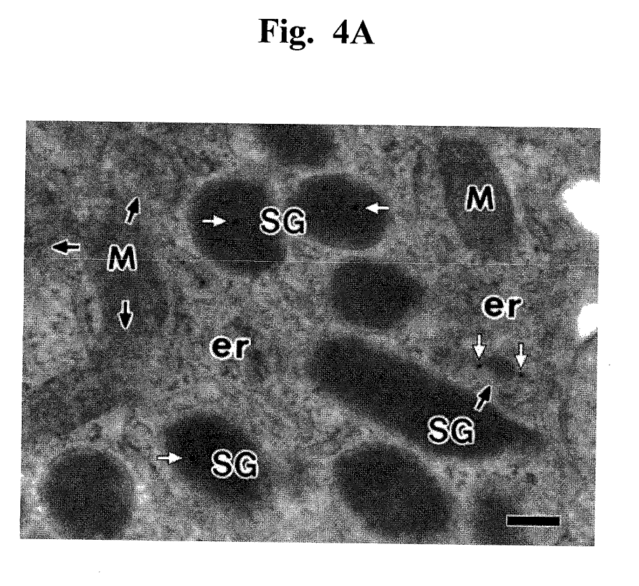

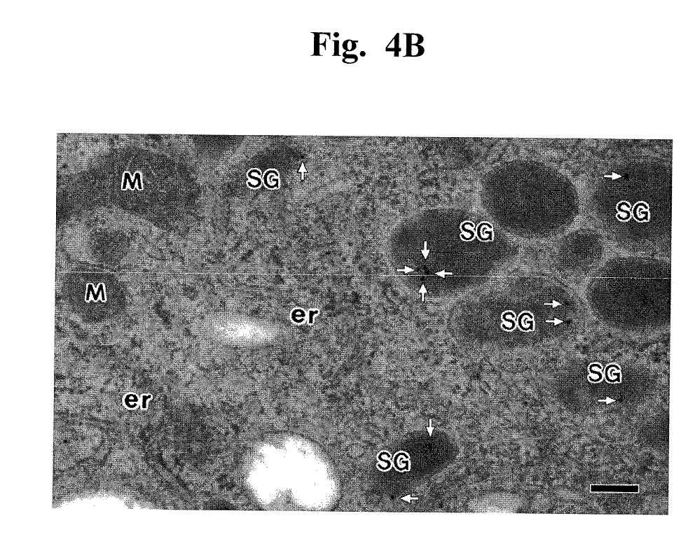

However, the results obtained from glioblastoma multiforme tissues were quite different from those of normal brain tissues. In stark contrast to the low number (0-3 per image) of secretory granules in astrocytes of normal brain tissues there were drastic increases in the number of secretory granule-like vesicles in both the cell body (FIG. 3A) and the processes (FIG. 3B) of astrocytes from GBM. The increase in the number of secretory granule-like vesicles in astrocytes of glioblastoma tissues was so dramatic that the cytoplasm of glioblastoma astrocytes in some images appeared to be full of secretory granule-like vesicles (Hg. 3, A and B). The identity of these secretory granule-like vesicles was again examined by the immunogold electron microscopy using the antibodies specific for chromogranin B (FIG. 4A) and secretogranin II (FIG. 4B). As shown in FIG. 4A, chromogranin B was present in the secretory granule-like vesicles in addition to its localization in the endoplasmic reticulum though it was absent in mitochondria. Likewise, secretogranin II was also localized in the secretory granule-like vesicles (FIG. 4B), but again was absent in mitochondria, thereby further confirming the identity of the secretory granule-like vesicles as secretory granules.

The distribution of the CGB- or SgII-labeling gold particles in the subcellular organelles in the astrocytes of both normal and glioblastoma brain tissues is summarized in Table 1. As shown in Table 1, the number of CGB-labeling gold particles per μm² of secretory granule area in normal astrocytes was 12.38 while that per μm² of mitochondria was 0.37, a background number, thus clearly demonstrating the presence of CGB in secretory granules. Likewise, the number of CGB-labeling gold particles per μm² of secretory granule area in GBM astrocytes was 11.98 while that per μm² of mitochondria was 0.36, further confirming the presence of CGB in secretory granules regardless of the pathogenic state of the astrocytes.

Analogous to CGB, the number of SgII-labeling gold particles per μm² of secretory granule area in normal astrocytes was 10.31 while that per μm² of mitochondria was 0.39 (Table 2), a background number, thereby clearly indicating the presence of SgII in secretory granules. Likewise, the number of SgII-labeling gold particles per μm² secretory granule area in GBM astrocytes was 8.70 while that per μm² of mitochondria was 0.31 (Table 2), further showing the presence of SgII in secretory granules of astrocytes regardless of the pathogenic state of the brain tissues. Considering the concentrated presence of the CGB- and SgII-labeling gold particles per unit area of secretory granules compared to that of the ER, secretory granules appeared to contain relatively large amounts of CGB and SgII, as was the case in chromaffin cells (Huh et al. 2005a), in both the normal and GBM astrocytes.

The number of and the area occupied by secretory granules in astrocytes from six different normal and six different GBM tissue samples are summarized in Table 3. Of the six normal and six GBM tissue samples that are used in the present study, in three cases both the normal and GBM tissue samples came from the same patients, but the rest (3 normal, 3 GBM) are not related to each other. Approximately a half of the cell images examined is the images that contain the cell body while the other half contains the cell processes. In normal astrocytes the number of secretory granules per cell image ranged 0.18-1.86, while the surface area of secretory granules per image ranged ˜0.03-0.16% of the total cell image area (Table 3). On the other hand, in GBM astrocytes the number of secretory granules per cell image was 15.0-22.89, and the surface area of secretory granules per image was ˜2.34-3.82% of the total cell image area.

TABLE 3
Distribution of secretory granules in astrocytes of normal and
glioblastoma multiforme human brain tissues.

| | | | | | | Sec- |
|---|---|---|---|---|---|---|
| | Tissue | | Sec- | | Number of | retory |
| | (number | Number | retory | | secretory | granule |
| | of cell | of | granule | | granules/ | area/cell |
| | images | secretory | area^{b} | Cell area | cell | area |
| Cell | used) | granules^{a} | (μm²) | (μm²) | image | (%) |
| Normal | 1 (11) | 2 | 0.262 | 852.058 | 0.16 | 0.03 |
| | 2 (8) | 5 | 0.343 | 715.088 | 0.63 | 0.05 |
| | 3 (10) | 4 | 0.930 | 1185.304 | 0.40 | 0.08 |
| | 4 (7) | 13 | 1.308 | 807.477 | 1.86 | 0.16 |
| | 5 (10) | 2 | 0.295 | 526.302 | 0.20 | 0.06 |
| | 6 (10) | 5 | 0.662 | 880.526 | 0.50 | 0.08 |
| GBM | 1 (13) | 245 | 21.126 | 570.518 | 18.85 | 3.70 |
| | 2 (10) | 150 | 14.488 | 382.306 | 15.00 | 3.38 |
| | 3 (11) | 194 | 12.364 | 529.390 | 17.64 | 2.34 |
| | 4 (11) | 173 | 21.021 | 651.772 | 15.73 | 3.22 |
| | 5 (9) | 193 | 14.901 | 398.316 | 21.44 | 3.74 |
| | 6 (9) | 206 | 16.662 | 436.021 | 22.89 | 3.82 |

^{a}The number of secretory granules in each picture image of normal and glioblastoma astrocytes ranged 0-3 and 11-46, respectively.
^{b}The area of secretory granules in each picture image of normal and glioblastoma astrocytes ranged 0-0.16% and 1.98-6.96%, respectively, of the total cell area.

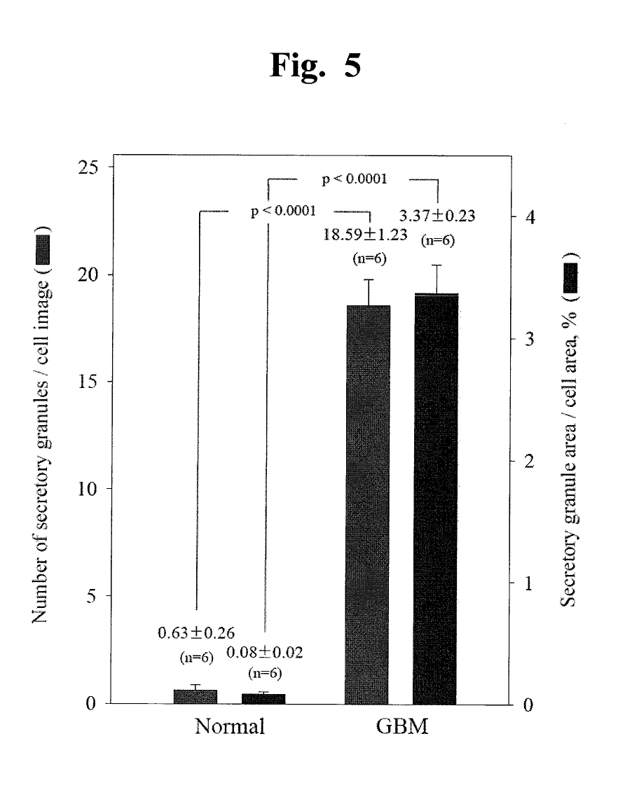

To obtain a better picture of the differences between the two groups the results in Table 3 are summarized in FIG. 5. The average number of secretory granules per cell image has increased 30-fold, changing from 0.63±0.26 (mean±s.e.) in normal cells to 18.59±1.23 (mean±s.e.) in GBM cells. Analogous to the increase in the number of secretory granules, the average surface area of secretory granules per cell image increased 42-fold, changing from 0.08±0.02 (mean±s.e.) in normal cells to 3.37±0.23 (mean±s.e.) in GBM cells. These results clearly show explosive increases in both the number of and the cell volume occupied by secretory granules in all cases of GBM astrocytes, although similar results were occasionally observed in lower grade tumors.

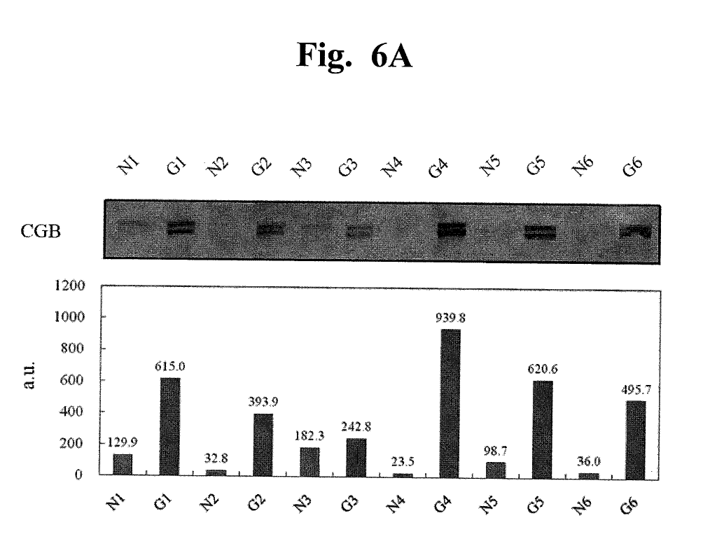

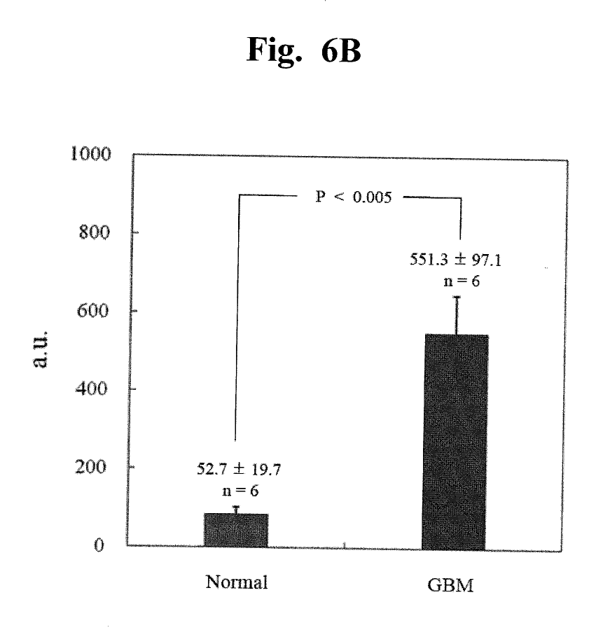

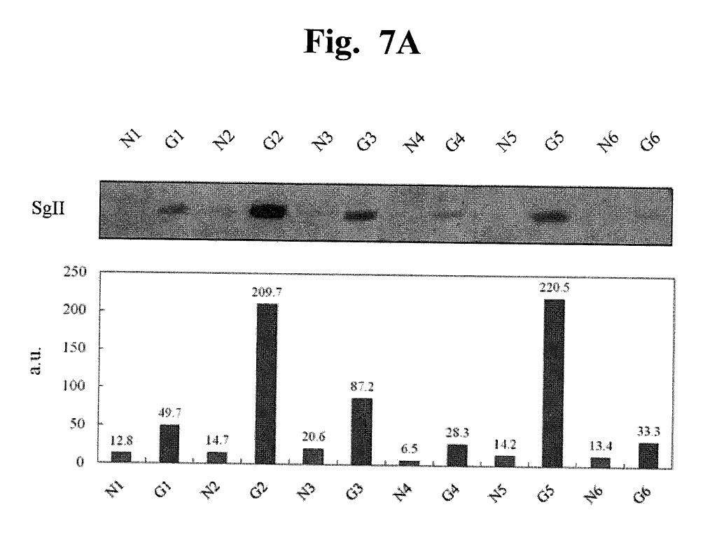

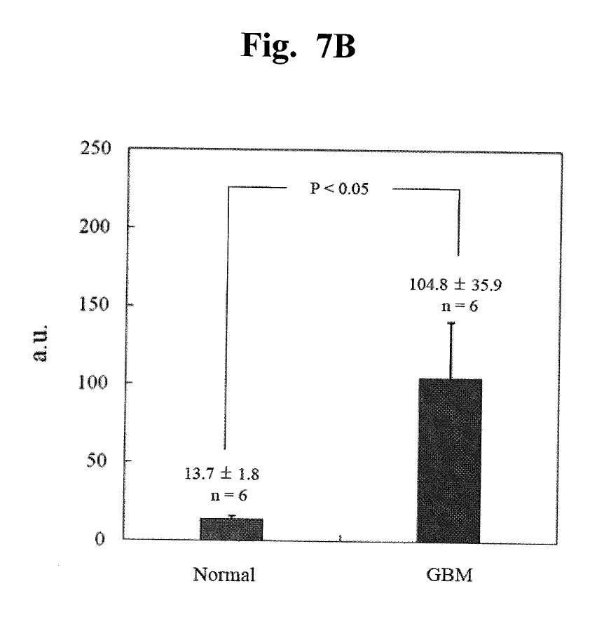

To determine whether the increase in the number of secretory granules can be detected by measuring the expression levels of secretory granule markers chromogranin B and secretogranin II, we have also examined the expression of CGB and SgII in the protein extracts of the normal and GBM tissues by immunoblot analysis (FIGS. 6 and 7). For this, we have chosen six normal and six GBM tissue samples that are not related to those of Table 3. As shown in FIG. 6A, chromogranin B is present in both the normal and GBM tissues, but the amounts of CGB expressed in glioblastoma tissues are significantly higher than those in normal tissues. The amounts of CGB expressed in GBM tissues are 2.7-80-fold (FIG. 6A), with an average of 10.5-fold (FIG. 6B), higher than those of normal tissues. Likewise, secretogranin II is also present in both the normal and GBM tissues (FIG. 7), and again the amounts of SgII expressed in the tumor tissues are far higher than those in normal tissues. The amounts of SgII expressed in GBM tissues are 2.5-16-fold (FIG. 7A), with an average of 7.6-fold (FIG. 7B), higher than those of normal tissues. These results indicate that the amounts of CGB and SgII expressed in glioblastoma tissues are 10.5-fold and 7.6-fold higher, respectively, than those in normal tissues, which are in accord with the increases in the number (30-fold) and the surface area (i.e., cell volume) (42-fold) of secretory granules produced in GBM astrocytes compared to those in normal astrocytes. The reason that the fold-increases of CGB and SgII expression in GBM tissues, ranging 7.6-10.5-fold, are lower than those of the number and the surface area of secretory granules, ranging 30-42-fold, of GBM astrocytes is understandable in light of the fact that the brain tissues from which the proteins are extracted consist of neurons and glial cells while the results obtained by electron microscopy are based exclusively on astrocytes of the brain tissues.

### Induction and Inhibition of Secretory Granule Formation in the Cell

Induction of Secretory Granules in Nonsecretory Cells that do not Normally contain Secretory Granules

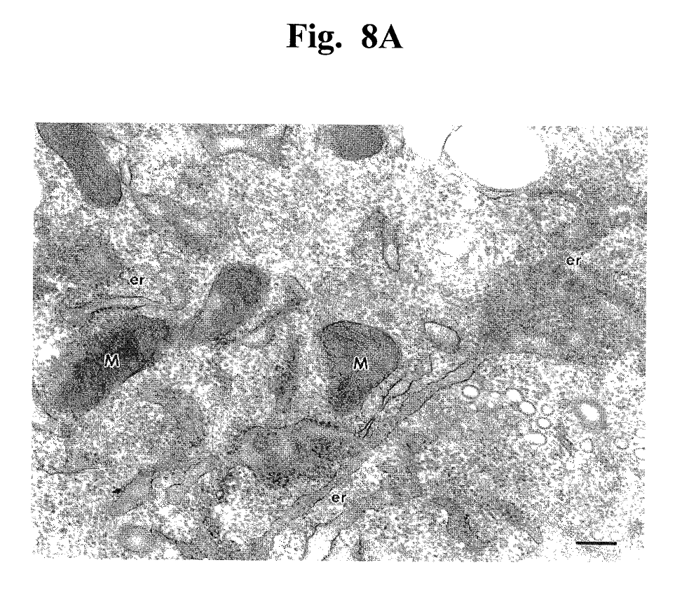

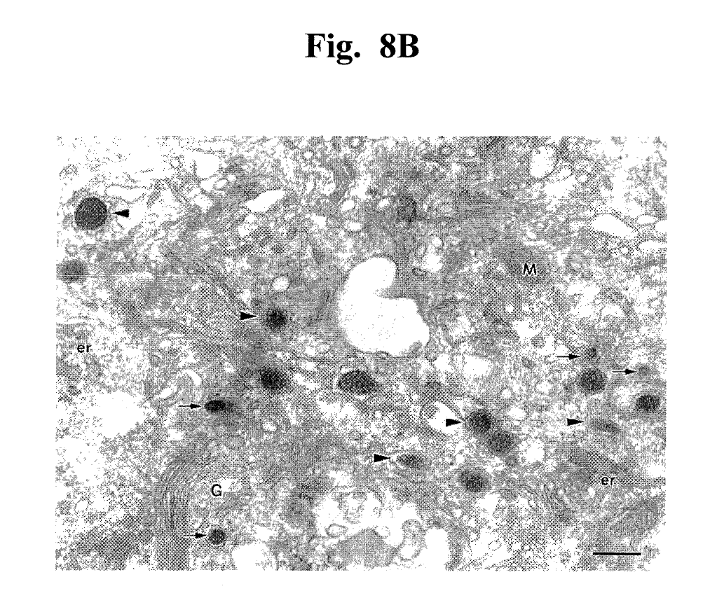

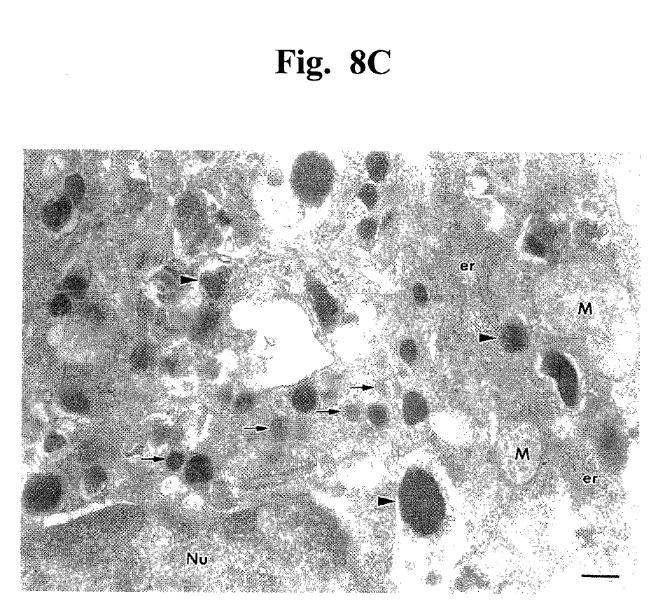

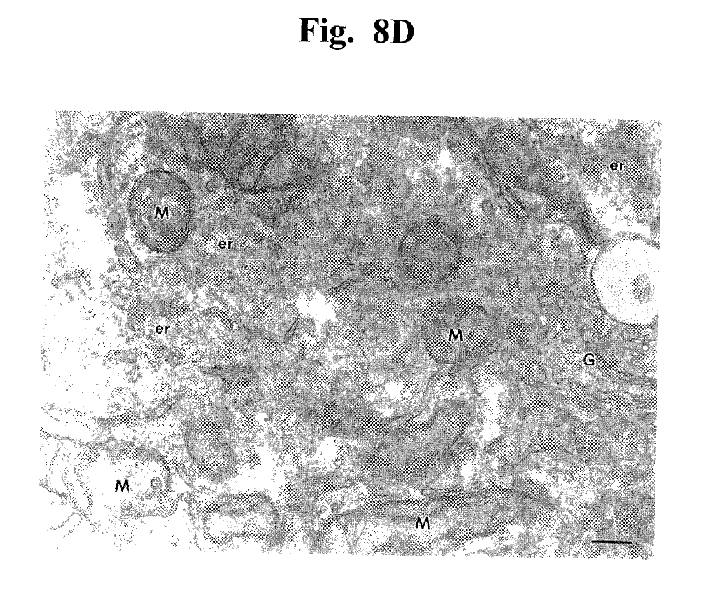

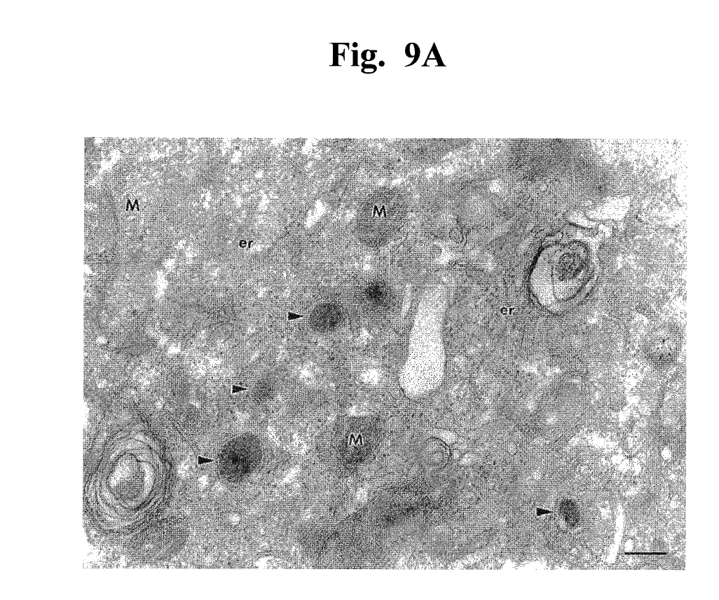

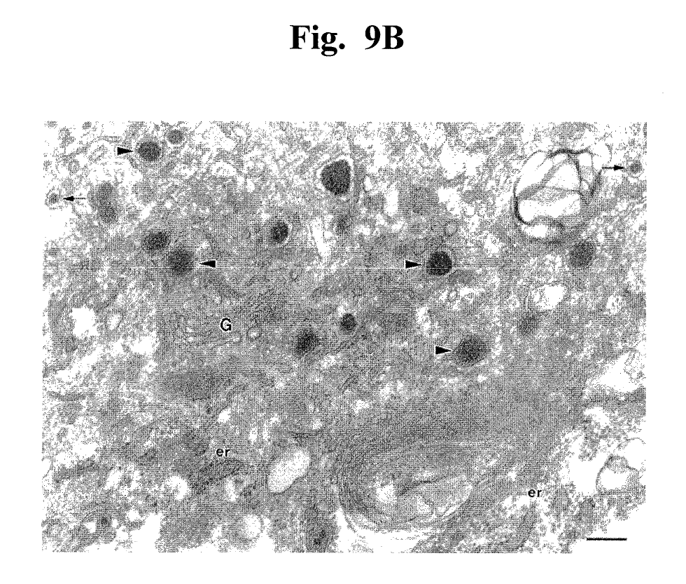

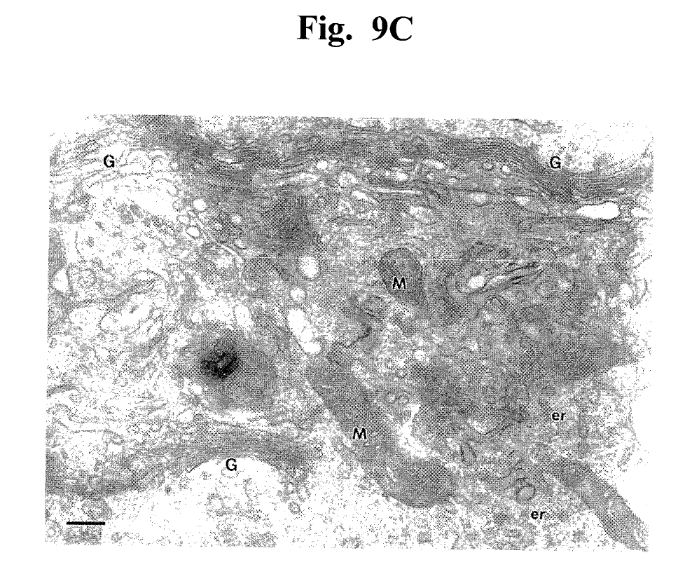

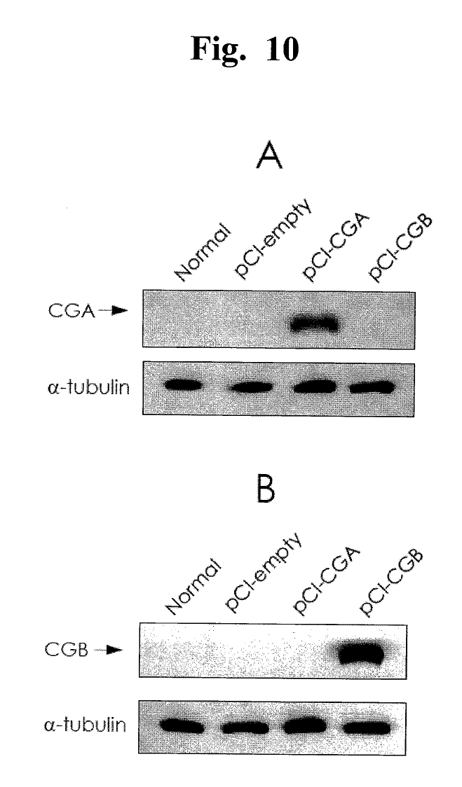

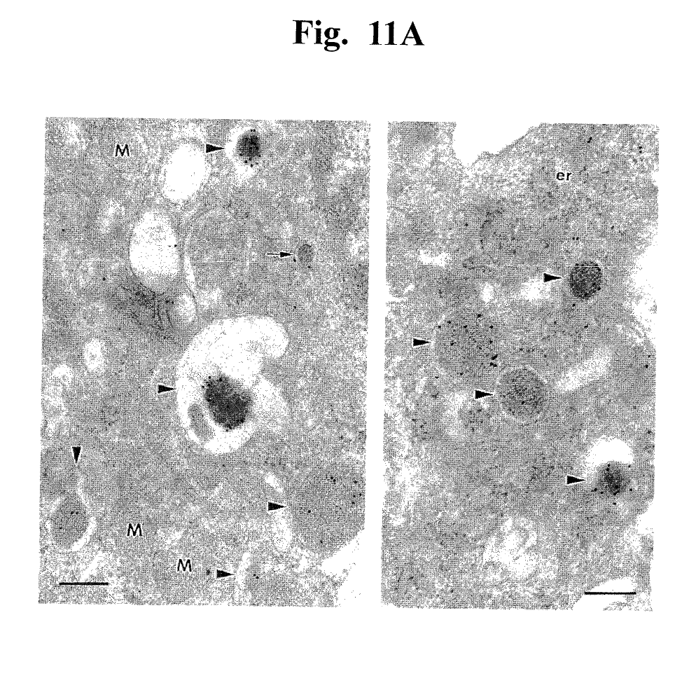

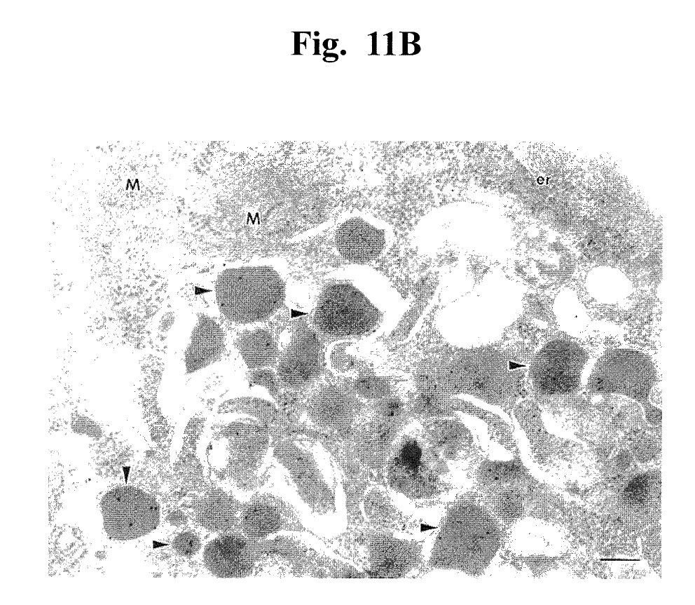

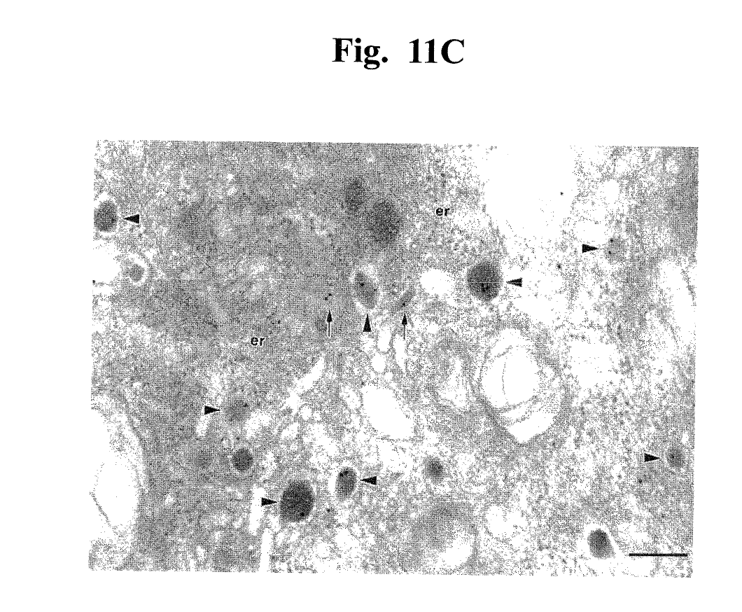

By expressing chromogranins A (CGA) and B (CGB) in nonsecretory cells such as NIH3T3 and COS-7 cells (FIGS. 8 and 9, Table 4), new secretory granules were formed in both NIH3T3 cells (FIG. 8) and COS-7 cells (FIG. 9) (Huh et al. 2003). But the number of secretory granules formed by CGB expression was ˜60% higher than those formed by CGA expression (Table 4), indicating the more potent granulogenic effect of chromogranin B (Huh et al. 2003). Transfection of CGA and CGB into NIH3T3 or COS-7 cells has been proven to express CGA and CGB, respectively, in the cells (FIGS. 10 and 11).

TABLE 4
Distribution of Dense-Core Secretory Granules in CGA- and CGB-
Transfected NIH3T3 and COS-7 Cells.

| | Normal | | Empty transfection | | CGA transfection | | CGB transfection | |
|---|---|---|---|---|---|---|---|---|
| | Number of | | Number of | | Number of | | Number of | |
| | granules/ | | granules/ | | granules/ | | granules/ | |
| | area | | area | | area | | area | |
| | viewed | granules/ | viewed | granules/ | viewed | granules/ | viewed | granules/ |
| | (μm²) | cell | (μm²) | cell | (μm²) | cell | (μm²) | cell |
| NIH3T3^{a} | 1/9130 | 0 | 14/9840 | 0.11 ± 0.32^{c} | 236/8205 | 2.51 ± 1.07^{d} | 317/7114 | 4.02 ± 0.75^{d} |
| COS-7^{b} | 1/20314 | 0 | 61/46556 | 0.10 ± 0.22^{c} | 596/41620 | 1.44 ± 0.89^{d} | 839/43271 | 2.23 ± 1.34^{d} |

^{a}70-78 cells sectioned from four different cell preparations were counted in each group.
^{b}150-300 cells sectioned from ten different cell preparations were counted in each group.
^{c}mean ± s.d.
^{d}mean ± s.d., p < 0.0001 by paired t-test.

Inhibition of Secretory Granules in Secretory Cells that Intrinsically Contain Secretory Granules

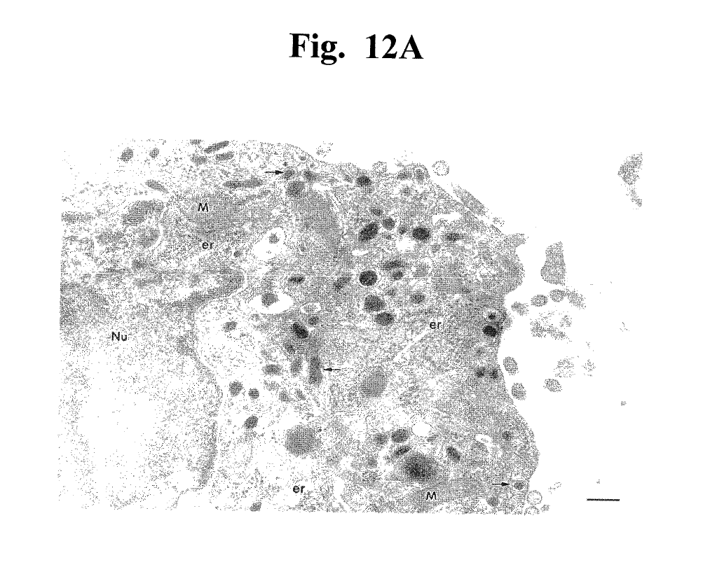

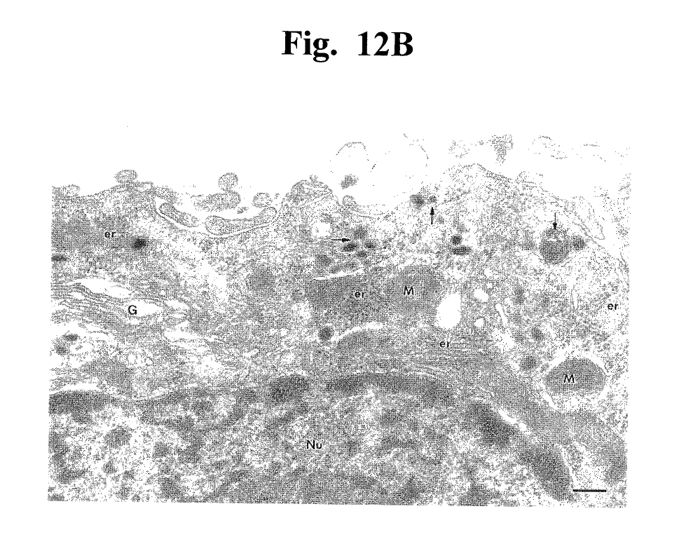

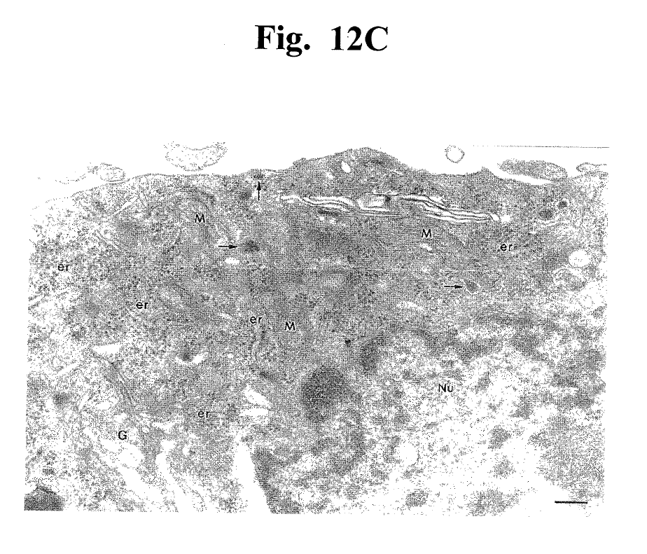

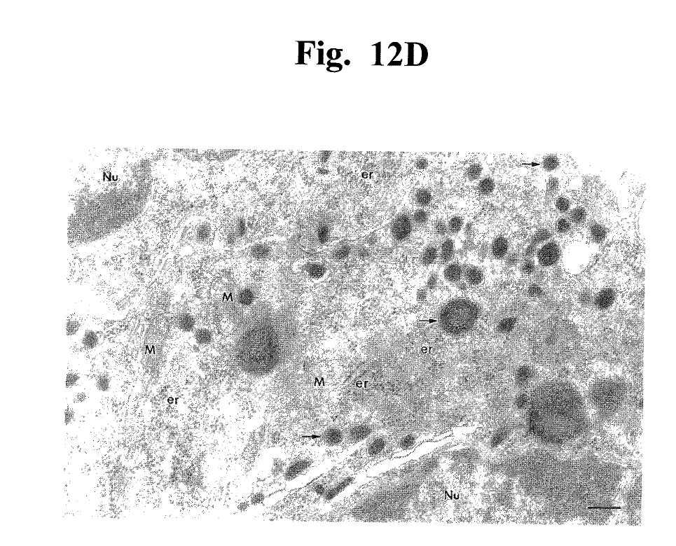

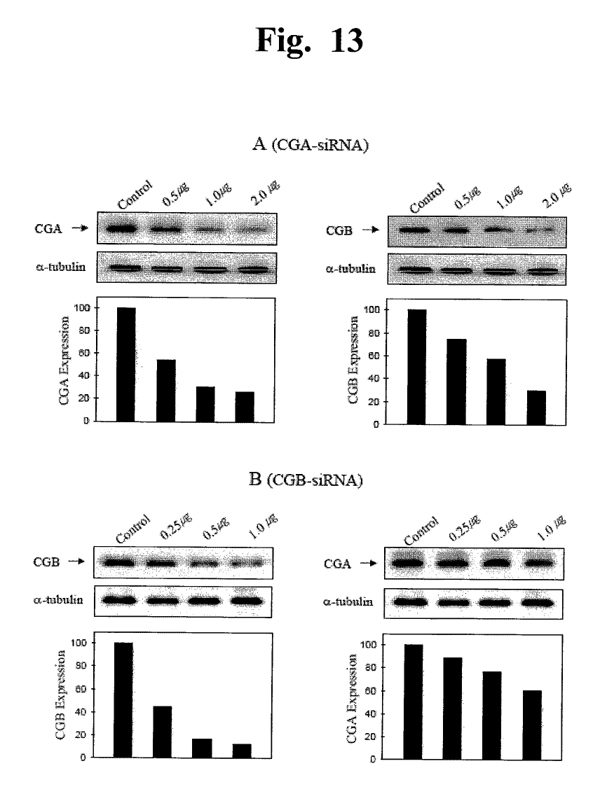

The production of secretory granules that exist in secretory cells intrinsically can also be suppressed by inhibiting the expression of chromogranins A and B. For example, the production of intrinsic secretory granules in typical secretory neuroendocrine PC12 cells was severely suppressed by inhibiting the expression of CGA or CGB in PC12 cells (FIG. 12, Table 5). The suppression of CGB expression decreased the number of secretory granules produced in PC12 cells by 78% while the suppression of CGA expression decreased the secretory granule production by 41% (FIG. 12, Table 5), demonstrating a significantly more potent effect of CGB. Suppression of chromogranin A or chromogranin B expression in PC12 cells was achieved by siRNA-CGA or siRNA-CGB treatment of PC12 cells (Huh et at. 2003). By the siRNA treatment the cognate chromogranin expression in the cells was reduced to 10-20% of the original level (FIG. 13).

TABLE 5
Distribution of Dense-Core Granules in CGA- and CGB-siRNA-Transfected PC12 Cells^{a}

| | | | | CGA-siRNA | | CGB-siRNA | |
|---|---|---|---|---|---|---|---|
| Normal PC12 cell | | Empty transfection | | transfection | | transfection | |
| Number of | | Number of | | Number of | | Number of | |
| granules/area | granules/ | granules/area | granules/ | granules/area | granules/ | granules/area | granules/ |
| viewed (μm²) | cell | viewed (μm²) | cell | viewed (μm²) | cell | viewed (μm²) | cell |
| 12244/3222 | 68.75 ± 8.50^{b} | 12504/3283 | 70.19 ± 13.80^{b} | 7015/3187 | 40.73 ± 7.49^{c} | 2632/3069 | 14.99 ± 6.30^{c} |

^{a}100 cells sectioned from four different cell preparations were counted in each group.
^{b}mean ± s.d.
^{c}mean ± s.d., p < 0.0001 by paired t-test.
^{d}A PC12 cell has 51-54 μm² of surface area in the central section that crosses the center of the nucleus, so the granules/cell indicates the total number of granules found divided by the respective average central-section area of a cell in each group.

### Discussion

In this regard, the present results that show the presence of chromogranin B and secretogranin II in the large dense-core vesicles in astrocytes of human brain tissues are in accord with the presence of secretory granules in human glial cells, and appear to underscore hitherto under-appreciated potential secretory activity of this organelle. It is therefore of great interest that the number of secretory granules in astrocytes of glioblastoma multiforme increased explosively (FIGS. 3 and 4, Table 3), as if to reveal a signature sign of the malignant brain tumor. The average number of secretory granules per cell image and the relative ratio of the secretory granule area over the total cell image area of the GBM astrocytes increased 30-fold and 42-fold, respectively, over the normal cells (Table 3 and FIG. 5). The stark contrast in the number of secretory granules between the normal and glioblastoma astrocytes has further been shown in the expression levels of chromogranin B and secretogranin II in the proteins that had been extracted from the normal and glioblastoma tissues (FIGS. 6 and 7), which showed 10.5-fold and 7.6-fold increases, respectively (FIGS. 6B and 7B). Considering that the brain tissues are composed of neurons and other glial cells such as oligodendrocytes and microglia, the 7.6-10.5-fold increase in the expression levels of CGB and SgII in the protein extracts still underscores the explosive increase in the number of granin-containing secretory granules in the astrocytes of glioblastoma multiforme.

Similar phenomena also occur in ganglioglioma, which is a tumor comprised of both neurons and glial cells, that there are also dramatic increases in the number of dense-core vesicles in the neoplastic neurons, particularly in the neuronal perikarya (Hirose et al. 1997; Sikorska et al. 2007). In addition, the expression of chromogranin A is also increased significantly in the neoplastic neurons of ganglioglioma (Hirose et al. 1997), thereby indicating the increase of secretory granules in these cells. It is further shown that the expression of neuropeptide Y, which is one of the integral components of secretory granules in both neurons and astrocytes, is abundant in the neoplastic neurons of ganglioglioma (Hirose et al. 1997). Given that chromogranins are granulogenic factors that induce formation of secretory granules in the cells they are expressed (Beuret et al. 2004; Huh et al. 2003; Kim et al. 2001), these results also indicate the increase in the number of secretory granules in the neoplastic neurons, thus strongly implicating the secretory granules to the neoplastic state of neurons.

The granin family proteins chromogranins A and B, and secretogranin II are the major proteins of secretory granules of neuroendocrine cells (Helle 2000; Montero-Hadjadje et al. 2008; Taupenot et al. 2003; Winkler and Fischer-Colbrie 1992; Huttner et al. 1991), and are high-capacity, low-affinity Ca²⁺ storage proteins (Yoo et al. 2001; Yoo and Albanesi 1991; Yoo et al. 2007). Due to the presence of the granin protein family, secretory granules contain the highest concentration of Ca^{2±}, ranging 20-40 mM (Haigh et al. 1989; Hutton 1989), among all the subcellular organelles in secretory cells. In addition, secretory granules also contain large amounts of the IP₃R/Ca²⁺ channels in their membranes (Huh et aZ 2005c; Yoo et al. 2001), which are directly bound to chromogranins A and B (Yoo et al. 2000; Yoo 2000). The bound chromogranins activate the IP₃R/Ca²⁺ channels (Yoo and Jeon 2000), increasing the channel open probability and the mean open time of the channels 8-16-fold and 9-42-fold, respectively (Thrower et al. 2002; Thrower et al., 2003).

Accordingly, secretory granules play a major role in IP₃-dependent intracellular Ca²⁺ control in secretory cells (Gerasimenko et al. 2006; Quesada et al. 2003; Quesada et al. 2001; Santodomingo et al. 2008; Srivastava et al. 1999; Xie et al. 2006; Gerasimenko et al, 1996; Nguyen et al. 1998; Yoo and Albanesi 1990): secretory granules have been shown to be responsible for >70% of IP₃-mediated Ca²⁺ mobilization in the cytoplasm of neuroendocrine cells (Huh et al. 2006; Huh et al. 2005b). Therefore, the presence of secretory granules in astrocytes not only indicates operation of secretory activity in these cells but also strongly points to the existence and operation of active IP₃-dependent Ca²⁺ storage and control mechanisms. Nevertheless, research on the functional significance of the presence of secretory granules in glial cells still remains very limited. In the present study we have shown that normal glial astrocytes express a few secretory granules in both the cell body and the processes though it appeared that more secretory granules are localized in the processes than in the cell body. However, in the glioblastoma astrocytes secretory granules appeared distributed widely in both the cell body and the processes (FIGS. 3, A and B), implying their universal roles in the cytoplasm of the affected cells.

Given that gliotransmitters such as glutamate, ATP, and peptides carry out essential roles in the cell-to-cell communication among the glial cells and neurons in the brain (Angulo et al. 2004; Fellin et al. 2006; Perea and Araque 2005; Volterra and Meldolesi 2005; Montana et al. 2006; Panatier et al. 2006; Haydon and Carmignoto 2006) and that the release of these gliotransmitters depends on the Ca²⁺ released from internal sources (Araque et al. 2000; Hua et al. 2004; Jeremic et al. 2001; Mothet et al. 2005; Martineau et al. 2008; Fellin et al. 2006; Santello and Volterra 2009; Ramamoorthy and Whim 2008), the extraordinary importance of intracellular Ca²⁺ stores in normal function of astrocytes becomes evident. That astrocytes lack the “active zones” that exist in neuronal synapse of neurons and adjoin voltage-gated Ca²⁺ channels further underscores the importance of the intracellular Ca²⁺ source in the control of many Ca²⁺-dependent activities of astrocytes (Santello and Volterra 2009; Haydon and Carmignoto 2006).

Moreover, the Ca²⁺-dependent glutamate release in astrocytes is shown to be due to the IP₃-dependent intracellular Ca⁺ releases (Araque et al. 2000; Hua et al. 2004; Jeremic et al. 2001). Therefore, considering that the release of Ca²⁺ from intracellular stores that leads to regulated secretory pathway is primarily linked to IP₃-dependent Ca²⁺ releases, it appears certain that the IP₃-sensitive intracellular Ca²⁺ stores of astrocytes play key roles in the normal functions of these cells (Hua et al. 2004; Jeremic et al. 2001; Araque et al. 2000). In this respect, participation of secretory granules both as the carrier of secretory contents and as an intracellular Ca²⁺ store appears to highlight the importance of secretory granules in the normal physiology of astrocytes. Hence, the number of organelles that can serve as the IP₃-sensitive intracellular Ca²⁺ stores will be critical in determining the Ca²⁺-dependent cellular activities of the cell in which they are localized, and for this reason the number of secretory granules present in the cell is likely to be a key determinant in the IP₃-dependent intracellular Ca²⁺ control of the cell (reviewed in Yoo, 2009).

Therefore, given that secretory granules are the major intracellular organelle that stores and controls intracellular Ca²⁺ concentration of the cell in which they are localized (Gerasimenko et al. 1996; Haigh et al. 1989; Huh et al. 2006; Huh et al. 2005b; Hutton 1989; Yoo and Albanesi 1990; Quesada et al. 2003; Nguyen et al. 1998), the presence of unusually large number of secretory granules in GBM astrocytes strongly implies critical roles of Ca²⁺ in the pathogenesis of brain tumors involving astrocytes. In this respect, it is conceivable that excessive availability of Ca²⁺ in astrocytes might not bode well for the well-being of the cells, and the change in the number of Ca²⁺-controlling secretory granules might have proportionately changed the overall capacity of the cells to control intracellular Ca²⁺ homeostasis. In view of the importance of Ca²⁺ in the control of a variety of cellular functions, the change in the overall capacity of cells to control intracellular Ca²⁺ homeostasis could easily affect differentiation of cells, potentially leading to development and proliferation of cancerous cells.

Taken together, we suggest that the number of secretory granules expressed in glial astrocytes has a direct relationship with the pathogenic state of human brain tissues. The abnormally high capacity of the affected astrocytes to store and release Ca²⁺ may play a major role in the pathogenesis of the brain tumor, resulting in a close correlation between the pathogenic state of GBM astrocytes and the increased number of secretory granules in the cells.

### REFERENCES

Angulo M. C., Kozlov A. S., Charpak S, and Audinat E. (2004) Glutamate released from glial cells synchronizes neuronal activity in the hippocampus. J. Neurosci. 24, 6920-6927.
Araque A., Li N., Doyle R. T. and Haydon P. G. (2000) SNARE protein-dependent glutamate release from astrocytes. J. Neurosci. 20, 666-673.
Ballut L, Marchadier B, Baguet A, Tomasetto C, Seraphin B, Le Hir H (2005) The exon junction core complex is locked onto RNA by inhibition of eIF4AIII ATPase activity. Nat Struct Mol Biol, 12, 861-869.
Beuret N., Stettler H., Renold A., Rutishauser J. and Spiess M. (2004) Expression of regulated secretory proteins is sufficient to generate granule-like structures in constitutively secreting cells. J. Biol. Chem. 279, 20242-20249.
Bezzi P., Gundersen V., Galbete 3. L., Seifert G., Steinhauser C., Pilati E. and Volterra A. (2004) Astrocytes contain a vesicular compartment that is competent for regulated exocytosis of glutamate. Nat. Neurosci. 7, 613-620.
Calegari F., Coco S., Taverna E., Bassetti M., Verderio C., Corradi N., Matteoli M. and Rosa P. (1999) A regulated secretory pathway in cultured hippocampal astrocytes. J. Biol. Chem. 274, 22539-22547.
Chen X., Wang L., Zhou Y., Zheng L. H. and Zhou Z. (2005) “Kiss-and-run” glutamate secretion in cultured and freshly isolated rat hippocampal astrocytes. J. Neurosci. 25, 9236-9243.
Coco S., Calegari F., Pravettoni E., Pozzi D., Taverna E., Rosa P., Matteoli M. and Verderio C. (2003) Storage and release of ATP from astrocytes in culture. J. Biol. Chem. 278, 1354-1362.
Coupland R. E. (1968) Determining sizes and distribution of sizes of spherical bodies such as chromaffin granules in tissue sections. Nature 217, 384-388.
Crippa D., Schenk U., Francolini M., Rosa P., Verderio C., Zonta M., Pozzan T., Matteoli M. and Carmignoto G. (2006) Synaptobrevin2-expressing vesicles in rat astrocytes: insights into molecular characterization, dynamics and exocytosis. J. Physiol. 570, 567-582.
Degot S, Regnier C H, Wendling C, Chenard M P, Rio M C, Tomasetto C (2002) Metastatic Lymph Node 51, a novel nucleo-cytoplasmic protein overexpressed in breast cancer. Oncogene, 21, 4422-4434.
Degot S, Le Hir H, Alpy F, Kedinger V, Stoll I, Wendling C, Seraphin B, R10 MC, Tomasetto C (2004) Association of the breast cancer protein MLN51 with the exon junction complex via its speckle localizer and RNA binding module. J Biol Chem, 279 33702-33715.
Fellin T., 5 ul J. Y., D'Ascenzo M., Takano H., Pascual O. and Haydon P. G. (2006) Bidirectional astrocyte-neuron communication: the many roles of glutamate and ATP. Novartis. Found. Symp. 276, 208-217.
Gerasimenko J. V., Sherwood M., Tepikin A. V., Petersen O. H. and Gerasimenko O. V. (2006) NAADP, cADPR and IP₃ all release Ca²⁺ from the endoplasmic reticulum and an acidic store in the secretory granule area. J. Cell Sci. 119, 226-238.
Gerasimenko O. V., Gerasimenko J. V., Belan P. V. and Petersen O. H. (1996) Inositol trisphosphate and cyclic ADP-ribose-mediated release of Ca²⁺ from single isolated pancreatic zymogen granules. Cell 84, 473-480.
Haigh J. R., Parris R. and Phillips J. H. (1989) Free concentrations of sodium, potassium and calcium in chromaffin granules. Biochem. J. 259, 485-491.
Haydon P. G. and Carmignoto G. (2006) Astrocyte control of synaptic transmission and neurovascular coupling. Physiol Rev. 86, 1009-1031.
Helle K. B. (2000) The chromogranins. Historical perspectives. Adv. Exp. Med. Biol. 482, 3-20.
Hirose T., Scheithauer B. W., Lopes M. B., Gerber H. A., Alternatt H. J. and VandenBerg S. R. (1997) Ganglioglioma: an ultrastructural and immunohistochemical study. Cancer 79, 989-1003.
Hua X., Malarkey E. B., Sunjara V., Rosenwald S. E., Li W. H. and Parpura V. (2004) Ca(2+)-dependent glutamate release involves two classes of endoplasmic reticulum Ca(2+) stores in astrocytes. J. Neurosci. Res. 76, 86-97.
Huh Y. H., Bahk S. J., Ghee J. Y. and Yoo S. H. (2005a) Subcellular distribution of chromogranins A and B in bovine adrenal chromaffin cells. FEBS Lett. 579, 5145-5151.
Huh Y. H., Chu S. Y., Park S. Y., Huh S. K. and Yoo S. H. (2006) Role of nuclear chromogranin B in inositol 1,4,5-trisphosphate-mediated nuclear Ca²⁺ mobilization. Biochemistry 45, 1212-1226.
Huh Y. H., Jeon S. H., Yoo J. A., Park S. Y. and Yoo S. H. (2005b) Effects of chromogranin expression on inositol 1,4,5-trisphosphate-induced intracellular Ca²⁺ mobilization. Biochemistry 44, 6122-6132.
Huh Y. H., Jeon S. H. and Yoo S. H. (2003) Chromogranin B-induced secretory granule biogenesis: comparison with the similar role of chromogranin A. J. Biol. Chem. 278, 40581-40589.
Huh Y. H., Yoo J. A., Bahk S. J. and Yoo S. H. (2005c) Distribution profile of inositol 1,4,5-trisphosphate receptor isoforms in adrenal chromaffin cells. FEES Lett. 579, 2597-2603.
Huttner W. B., Gerdes H. H. and Rosa P. (1991) The granin (chromogranin/secretogranin) family. Trends Biochem. Sci. 16, 27-30.
Hutton J. C. (1989) The insulin secretory granule. Diabetologia 32, 271-281. Jeremic A., Jeftinija K., Stevanovic J., Glavaski A. and Jeftinija S. (2001) ATP stimulates calcium-dependent glutamate release from cultured astrocytes. J. Neurochem. 77, 664-675.
Kim T., Tao-Cheng 3. H., Eiden L. E. and Loh Y. P. (2001) Chromogranin A, an “on/off” switch controlling dense-core secretory granule biogenesis. Cell 106, 499-509.
Kreft M., Stenovec M., Rupnik M., Grilc S., Krzan M., Potokar M., Pangrsic T., Haydon P. G. and Zorec R. (2004) Properties of Ca(2+)-dependent exocytosis in cultured astrocytes. Glia 46, 437-445.
Krzan M., Stenovec M., Kreft M., Pangrsic T., Grilc S., Haydon P. G. and Zorec R. (2003) Calcium-dependent exocytosis of atrial natriuretic peptide from astrocytes. J. Neurosci. 23, 1580-1583.
Maienschein V., Marxen M., Volknandt W. and Zimmermann H. (1999) A plethora of presynaptic proteins associated with ATP-storing organelles in cultured astrocytes. Glia 26, 233-244.
Martineau M., Galli T., Baux G. and Mothet J. P. (2008) Confocal imaging and tracking of the exocytotic routes for D-serine-mediated gliotransmission. Glia 56, 1271-1284.
McAuliffe W. G. and Hess A. (1990) Human chromogranin A-like immunoreactivity in the Bergmann glia of the rat brain. Gila 3, 13-16.
Montana V., Malarkey E. B., Verderio C., Matteoli M. and Parpura V. (2006) Vesicular transmitter release from astrocytes. Glia 54, 700-715.
Montana V., Ni Y., Sunjara V., Hua X. and Parpura V. (2004) Vesicular glutamate transporter-dependent glutamate release from astrocytes. J. Neurosci. 24, 2633-2642.
Montero-Hadjadje M., Vaingankar S., Elias S., Tostivint H., Mahata S. K. and Anouar Y. (2008) Chromogranins A and B and secretogranin II: evolutionary and functional aspects. Acta Physiol (Oxf) 192, 309-324.
Mothet 3. P., Pollegioni L., Ouanounou G., Martineau M., Fossier P. and Baux G. (2005) Glutamate receptor activation triggers a calcium-dependent and SNARE protein-dependent release of the gliotransmitter D-serine. Proc. Natl. Acad. Sci. U.S. A 102, 5606-5611.
Nguyen T., Chin W. C. and Verdugo P. (1998) Role of Ca²⁺/K⁺ ion exchange in intracellular storage and release of Ca²⁺. Nature 395, 908-912.
Panatier A., Theodosis D. T., Mothet J. P., Touquet B., Pollegioni L., Poulain D. A. and Oliet S. H. (2006) Glia-derived D-serine controls NMDA receptor activity and synaptic memory. Cell 125, 775-784.
Park H. Y., So S. H., Lee W. B., You S. H. and Yoo S. H. (2002) Purification, pH-dependent conformational change, aggregation, and secretory granule membrane binding property of secretogranin II (chromogranin C). Biochemistry 41, 1259-1266.
Parpura V. and Haydon P. G. (2000) Physiological astrocytic calcium levels stimulate glutamate release to modulate adjacent neurons. Proc. Natl. Acad. Sci. U.S.A 97, 8629-8634.
Perea G. and Araque A. (2005) Glial calcium signaling and neuron-glia communication. Cell Calcium 38, 375-382.
Potokar M., Stenovec M., Kreft M., Kreft M. E. and Zorec R. (2008) Stimulation inhibits the mobility of recycling peptidergic vesicles in astrocytes. Glia 56, 135-144. Quesada I., Chin W. C., Steed J., Campos-Bedolla P. and Verdugo P. (2001) Mouse mast cell secretory granules can function as intracellular ionic oscillators. Biophys. J. 80, 2133-2139.
Quesada I., Chin W. C. and Verdugo P. (2003) ATP-independent luminal oscillations and release of Ca²⁺ and H⁺ from mast cell secretory granules: implications for signal transduction. Biophys. J. 85, 963-970.
Ramamoorthy P. and Whim M. D. (2008) Trafficking and fusion of neuropeptide Y-containing dense-core granules in astrocytes. J. Neurosci. 28, 13815-13827.
Santello M. and Volterra A. (2009) Synaptic modulation by astrocytes via Ca²⁺-dependent glutamate release. Neuroscience 158, 253-259.
Santodomingo J., Vay L., Camacho M., Hernandez-SanMiguel E., Fonteriz R. I., Lobaton C. D., Montero M., Moreno A. and Alvarez J. (2008) Calcium dynamics in bovine adrenal medulla chromaffin cell secretory granules. Eur. J. Neurosci. 28, 1265-1274.
Sikorska B., Papierz W., Zakrzewki K., Fiks T., Polis L. and Liberski P. P. (2007) Ultrastructural heterogeneity of gangliogliomas. Ultrastruct. Pathol. 31, 9-14.
Srivastava M., Atwater I., Glasman M., Leighton X., Goping G., Caohuy H., Miller G., Pichel J., Westphal H., Mears D., Rojas E. and Pollard H. B. (1999) Defects in inositol 1,4,5-trisphosphate receptor expression, Ca²⁺ signaling, and insulin secretion in the anx7(+/−) knockout mouse. Proc. Natl. Acad. 5ci. U.S. A 96, 13783-13788.
Striedinger K., Meda P. and Scemes E. (2007) Exocytosis of ATP from astrocyte progenitors modulates spontaneous Ca²⁺ oscillations and cell migration. Glia 55, 652-662.
Taupenot L., Harper K. L. and O'Connor D. T. (2003) The chromogranin-secretogranin family. N. Engl. J. Med. 348, 1134-1149.
Thrower E. C., Choe C. U., So S. H., Jeon S. H., Ehrlich B. E. and Yoo S. H. (2003) A functional interaction between chromogranin B and the inositol 1,4,5-trisphosphate receptor/Ca²⁺ channel. J. Biol. Chem. 278, 49699-49706.
Thrower E. C., Park H. Y., So S. H., Yoo S. H. and Ehrlich B. E. (2002) Activation of the inositol 1,4,5-trisphosphate receptor by the calcium storage protein chromogranin A. J. Biol. Chem. 277, 15801-15806.
Volterra A. and Meldolesi 3. (2005) Astrocytes, from brain glue to communication elements: the revolution continues. Nat. Rev. Neurosa, 6, 626-640.
Winkler H. and Fischer-Colbrie R. (1992) The chromogranins A and B: the first 25 years and future perspectives. Neuroscience 49, 497-528.
Xie L., Zhang M., Zhou W., Wu Z., Ding J., Chen L. and Xu T. (2006) Extracellular ATP Stimulates Exocytosis via Localized Ca Release from Acidic Stores in Rat Pancreatic beta Cells. Traffic. 7, 429-439.
Yoo S. H. (1995) Purification and pH-dependent secretory vesicle membrane binding of chromogranin B. Biochemistry 34, 8680-8686.
Yoo S. H. (2000) Coupling of the IP₃ receptor/Ca²⁺ channel with Ca²⁺ storage proteins chromogranins A and B in secretory granules. Trends Neurosci. 23, 424-428.
Yoo S. H. (2009) Secretory granules in inositol 1,4,5-trisphosphate-dependent Ca²⁺ signaling in the cytoplasm of neuroendocrine cells. FASEB J. online published Oct. 16, 2009
Yoo S. H. and Albanesi 3. P. (1991) High capacity, low affinity Ca²⁺binding of chromogranin A. Relationship between the pH-induced conformational change and Ca²⁺ binding property. J. Biol. Chem. 266, 7740-7745.
Yoo S. H. and Albanesi 3. P. (1990) Inositol 1,4,5-trisphosphate-triggered Ca²⁺ release from bovine adrenal medullary secretory vesicles. J. Biol. Chem. 265, 13446-13448.
Yoo S. H., Chu S. Y., Kim K. D. and Huh Y. H. (2007) Presence of secretogranin II and high-capacity, low-affinity Ca²⁺ storage role in nucleoplasmic Ca²⁺ store vesicles. Biochemistry 46, 14663-14671.

Yoo S. H. and Jeon C. J. (2000) Inositol 1,4,5-trisphosphate receptor/Ca²⁺ channel modulatory role of chromogranin A, a Ca²⁺ storage protein of secretory granules. J. Biol. Chem. 275, 15067-15073.
Yoo S. H., Oh Y. S., Kang M. K., Huh Y. H., So S. H., Park H. S, and Park H. Y. (2001) Localization of three types of the inositol 1,4,5-trisphosphate receptor/Ca²⁺ channel in the secretory granules and coupling with the Ca²⁺ storage proteins chromogranins A and B. J. Biol. Chem. 276, 45806-45812.
Yoo S. H., So S. H., Kweon H. S., Lee J. S., Kang M. K. and Jeon C. J. (2000)

Coupling of the inositol 1,4,5-trisphosphate receptor and chromogranins A and B in secretory granules. J. Biol. Chem. 275, 12553-12559.
Yoo S. H., You S. H., Kang M. K., Huh Y. H. and Shim C. S. (2002) Localization of secretory granule marker protein chromogranin B in the nucleus. Ann. N.Y. Acad. Sa. 971, 345-348.

Having described a preferred embodiment of the present invention, it is to be understood that variants and modifications thereof falling within the spirit of the invention may become apparent to those skilled in this art, and the scope of this invention is to be determined by appended claims and their equivalents.

1.-27. (canceled)

28. A method for determining whether a test subject has cancer, the methods comprising the steps of:
(a) contacting a sample from the subject with a binding agent that specifically binds to a granulogenic factor or to a nucleic acid molecule encoding said granulogenic factor, and
(b) determining the level of binding of the binding agent to said granulogenic factor or said nucleic acid molecule to determine whether the test subject has cancer.

29. The method according to claim 28, wherein the granulogenic factor comprises chromogranins or secretogranins.

30. The method according to claim 29, wherein the granulogenic factor comprises chromogranin B (CGB) or secretogranin II (SgII).

31. The method according to claim 28, wherein the cancer is selected from the group consisting of brain cancer, neuroendocrine cancer, stomach cancer, lung cancer, breast cancer, ovarian cancer, liver cancer, nasopharyngeal cancer, laryngeal cancer, pancreatic cancer, bladder cancer, adrenal cancer, colon cancer, colorectal cancer, cervical cancer, prostate cancer, bone cancer, skin cancer, thyroid cancer, parathyroid cancer and ureter cancer.

32. The method according to claim 31, wherein the cancer is a secretory cell tumor.

33. The method according to claim 32, wherein the secretory cell tumor comprises brain cancer, neuroendocrine cancer, ganglioglioma, pituitary adenoma, adrenal cancer, breast cancer, cervical cancer or prostate cancer.

34. The method according to claim 28, wherein the binding agent comprises an antibody or aptamer.

35. The method according to claim 28, wherein the binding agent comprises a hybridization probe or primer

36. The method of according to claim 28, wherein detection of an increase in level of binding relative to a normal control indicates the identification of cancer in the subject.


SEQUENCE LISTING (6 sequences)

SEQ ID NO: 1 - DNA, 1374 bp, Homo sapiens
atgcgctccgccgctgtcctggctcttctgctctgcgccgggcaagtcactgcgctccctgtgaacagccctatgaataaaggggataccgaggtgatgaaatgcatcgttgaggtcatctccgacacactttccaagcccagccccatgcctgtcagccaggaatgttttgagacactccgaggagatgaacggatcctttccattctgagacatcagaatttactgaaggagctccaagacctcgctctccaaggcgccaaggagagggcacatcagcagaagaaacacagcggttttgaagatgaactctcagaggttcttgagaaccagagcagccaggccgagctgaaagaggcggtggaagagccatcatccaaggatgttatggagaaaagagaggattccaaggaggcagagaaaagtggtgaagccacagacggagccaggccccaggccctcccggagcccatgcaggagtccaaggctgaggggaacaatcaggcccctggggaggaagaggaggaggaggaggaggccaccaacacccaccctccagccagcctccccagccagaaatacccaggcccacaggccgagggggacagtgagggcctctctcagggtctggtggacagagagaagggcctgagtgcagagccagggtggcaggcaaagagagaagaggaggaggaggaggaggaggaggctgaggctggagaggaggctgtccccgaggaagaaggccccactgtagtgctgaacccccacccgagccttggctacaaggagatccggaaaggcgagagtcggtcggaggctctggctgtggatggagctgggaagcctggggctgaggaggctcaggaccccgaagggaagggagaacaggagcactcccagcagaaagaggaggaggaggagatggcagtggtcccgcaaggcctcttccggggtgggaagagcggagagctggagcaggaggaggagcggctctccaaggagtgggaggactccaaacgctggagcaagatggaccagctggccaaggagctgacggctgagaagcggctggaggggcaggaggaggaggaggacaaccgggacagttccatgaagctctccttccgggcccgggcctacggcttcaggggccctgggccgcagctgcgacgaggctggaggccatcctcccgggaggacagccttgaggcgggcctgcccctccaggtccgaggctaccccgaggagaagaaagaggaggagggcagcgcaaaccgcagaccagaggaccaggagctggagagcctgtcggccattgaagcagagctggagaaagtggcccaccagctgcaggcactacggcggggctga

SEQ ID NO: 2 - protein, 457 aa, Homo sapiens
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 3 - DNA, 2034 bp, Homo sapiens
atgcagccaacgctgcttctcagcctcctgggagccgtggggctggcggctgtcaattccatgccagtggataacaggaaccacaatgaaggaatggtgactcgctgcatcattgaggtcctctcaaatgccttgtcgaagtccagcgctccacccatcacccctgagtgccgccaagtcctgaagacgagtagaaaagacgtcaaagacaaagagacaactgaaaatgaaaacacaaagtttgaagtaagattgttaagagacccagctgatgcctcggaagcccacgagtcctccagcaggggagaggcaggagccccaggggaggaggacatccaaggcccaacaaaggcagacacagagaaatgggcagagggaggcgggcacagccgagagcgagcggatgagccccagtggagcctctatccctccgacagccaagtctctgaagaagtgaagacacgccattctgagaagagccagagagaggatgaggaggaggaggagggagagaactatcaaaaaggggagcgaggggaagatagcagtgaagagaaacaccttgaagagccaggagagacacaaaacgcttttctcaatgaaagaaagcaggcttcagctataaaaaaagaggagttagtggccagatcggaaacacatgctgccgggcattctcaggagaagacacatagccgagagaagagtagccaggagagtggagaggagacagggagccaggagaatcacccccaggagtctaaaggccaaccccgaagccaggaagaatctgaggaaggtgaggaagatgccacctctgaggtggacaaacgacgcacgaggcccagacaccaccacgggaggagcaggcccgacaggtcctctcaaggagggagtcttccctctgaggaaaagggacacccccaggaggaatctgaggagtcaaacgtcagcatggccagtttaggggaaaagagggaccaccattcaacccactacagggcttcagaggaagaacctgaatatggagaagaaataaagggttatccaggcgtccaggcccctgaggacctggagtgggagcgctataggggcagaggaagtgaagaatacagggctccaagacctcagagtgaggagagttgggatgaggaggacaagagaaactaccccagcttagagcttgataagatggcacatggatatggtgaagaaagtgaggaagagaggggccttgagccgggaaagggacgccatcacagaggcaggggaggggagccacgtgcctatttcatgtctgacaccagagaagagaaaaggttcttgggtgaaggacaccaccgtgtccaagaaaaccagatggacaaggcaaggaggcatccacaaggtgcgtggaaagagctggacagaaattatctcaactacggtgaggaaggagccccagggaagtggcagcagcagggagacctgcaggacactaaagaaaacagggaggaagctaggtttcaagataaacaatatagctcccatcacacagctgaaaagaggaagagattaggggaactgttcaacccatactacgaccctctccagtggaagagcagccattttgaaagaagagacaacatgaatgacaattttctcgagggtgaggaggaaaatgagctgaccttgaacgagaagaatttcttcccagaatacaactatgactggtgggagaaaaagcccttctctgaggatgtgaactgggggtatgagaagagaaacctcgccagggtccccaagctggacctgaaaaggcaatatgacagggtggcccaactggaccagctccttcactacaggaagaagtcagctgagtttccagacttctatgattctgaggagccggtgagcacccaccaggaggcagaaaatgaaaaggacagggctgaccagacagtcctgacagaggacgagaaaaaagaactcgaaaacttggctgcaatggatttggaactacagaagatagctgagaaattcagccaaaggggctga

SEQ ID NO: 4 - protein, 677 aa, Homo sapiens
MQPTLLLSLLGAVGLAAVNSMPVDNRNHNEGMVTRCIIEVLSNALSKSSAPPITPECRQVLKTSRKDVKDKETTENENTKFEVRLLRDPADASEAHESSSRGEAGAPGEEDIQGPTKADTEKWAEGGGHSRERADEPQWSLYPSDSQVSEEVKTRHSEKSQREDEEEEEGENYQKGERGEDSSEEKHLEEPGETQNAFLNERKQASAIKKEELVARSETHAAGHSQEKTHSREKSSQESGEETGSQENHPQESKGQPRSQEESEEGEEDATSEVDKRRTRPRHHHGRSRPDRSSQGGSLPSEEKGHPQEESEESNVSMASLGEKRDHHSTHYRASEEEPEYGEEIKGYPGVQAPEDLEWERYRGRGSEEYRAPRPQSEESWDEEDKRNYPSLELDKMAHGYGEESEEERGLEPGKGRHHRGRGGEPRAYFMSDTREEKRFLGEGHHRVQENQMDKARRHPQGAWKELDRNYLNYGEEGAPGKWQQQGDLQDTKENREEARFQDKQYSSHHTAEKRKRLGELFNPYYDPLQWKSSHFERRDNMNDNFLEGEEENELTLNEKNFFPEYNYDWWEKKPFSEDVNWGYEKRNLARVPKLDLKRQYDRVAQLDQLLHYRKKSAEFPDFYDSEEPVSTHQEAENEKDRADQTVLTEDEKKELENLAAMDLELQKIAEKFSQRG

SEQ ID NO: 5 - DNA, 1854 bp, Homo sapiens
atggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataaggaagaaagcagcccagattataatccctaccaaggtgtctctgtcccccttcagcaaaaagaaaatggcgatgaaagccacttgcccgagagggattcactgagtgaagaagactggatgagaataatactcgaagctttgagacaggctgaaaatgagcctcagtctgcaccaaaagaaaataagccctatgccttgaattcagaaaagaactttccaatggacatgagtgatgattatgagacacagcagtggccagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatatagaaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaaccaagaaaaggcagaaaagggaagggagcatattgctaagagagcaatggaaaatatgtaa

SEQ ID NO: 6 - protein, 617 aa, Homo sapiens
MAEAKTHWLGAALSLIPLIFLISGAEAASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM